(19) United States
(12) Patent Application Publication
(10) Pub. No.: US 2025/0226104 A1
(43) Pub. Date: Jul. 10, 2025

(54) DETECTION OF SEPSIS USING HEMATOLOGY PARAMETERS

(71) Applicants: Beckman Coulter, Inc., Brea, CA (US); The Johns Hopkins University, Baltimore, MD (US)

(72) Inventors: Scott R. LEVIN, Washington, DC (US); Jeremiah HINSON, Baltimore, MD (US); Aria G. SMITH, Baltimore, MD (US); Matthew TOERPER, Cockeysville, MD (US); Oluwakemi BADAKI-MAKUN, Baltimore, MD (US); Richard ROTHMAN, Cockeysville, MD (US); Sarah Michelle KEHOE, Reading, MA (US); Arnaud DEBRAINE, Brea, CA (US)

(21) Appl. No.: 18/839,404

(22) PCT Filed: Feb. 17, 2023
(86) PCT No.: PCT/US2023/062832

§ 371 Date: Aug. 16, 2024

Related U.S. Application Data
(60) Provisional application No. 63/311,600, filed on Feb. 18, 2022.

(51) Int. Cl. G16H 50/30; G01N 15/10; G01N 15/14; G01N 15/1429; G01N 27/02; G16H 15/00; G16H 20/10; G16H 50/70

(52) U.S. Cl. CPC G16H 50/30; G01N 15/1429; G01N 15/1459; G01N 27/02; G16H 15/00; G16H 20/10; G16H 50/70; G01N 2015/1006

(57) ABSTRACT

This disclosure describes methods of and systems for screening for sepsis or septic shock in a patient and methods of ruling out sepsis or septic shock in a patient using white blood cell count (WBC), a monocyte cell population parameter, or neutrophil-to-lymphocyte ratio (NLR), or a combination thereof, in the blood sample from the patient.

### CROSS REFERENCE TO RELATED APPLICATIONS

This application is a National Stage Entry of PCT Application No. PCT/US2023/062832, entitled “Detection of Sepsis Using Hematology Parameters,” filed Feb. 17, 2023, which is related to, and claims the benefit of, provisional patent application 63/311,600, titled “Detection of Sepsis Using Hematology Parameters,” filed in the United States Patent Office on Feb. 18, 2022. Those applications are hereby incorporated by reference in their entirety for all purposes.

### GOVERNMENT FUNDING

This invention was made with government support under HS026640-02 awarded by U.S. Department of Health and Human Services. The government has certain rights in the invention.

### BACKGROUND

Sepsis is a leading cause of morbidity and mortality worldwide and is responsible for over 1.5 million hospitalizations and 250,000 deaths in the United States each year. Early initiation of targeted treatments for sepsis improves patient outcomes and lower costs, but reliable identification of sepsis remains challenging.

### SUMMARY OF THE INVENTION

In one aspect, this disclosure describes a method of screening for sepsis or septic shock in a patient. The method includes calculating neutrophil-to-lymphocyte ratio (NLR) for the blood sample from the patient; characterizing white blood cell count (WBC) for a blood sample; calculating a monocyte cell population parameter for the blood sample; comparing NLR to a first predetermined set of threshold values, WBC to a second predetermined set of threshold values, and the monocyte cell population parameter to a third predetermined set of threshold values; and identifying the patient on a spectrum of low to high risk of sepsis or septic shock. In some embodiments, the method includes reporting the patient's identified risk to a clinician in a view within the electronic medical record (EMR).

The words “preferred” and “preferably” refer to embodiments or aspects of the invention that may afford certain benefits, under certain circumstances. However, other embodiments or aspects may also be preferred, under the same or other circumstances. Furthermore, the recitation of one or more preferred embodiments or aspects does not imply that other embodiments or aspects are not useful and is not intended to exclude other embodiments or aspects from the scope of the invention.

The term “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Unless otherwise specified, “a,” “an,” “the,” and “at least one” are used interchangeably and mean one or more than one.

As used herein, the term “or” is generally employed in its usual sense including “and/or” unless the content clearly dictates otherwise.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

Herein, “up to” a number (for example, up to 50) includes the number (for example, 50).

The term “in the range” or “within a range” (and similar statements) includes the endpoints of the stated range.

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

Reference throughout this specification to “one embodiment,” “one aspect,” “an embodiment,” “an aspect,” “certain embodiments,” “certain aspects, “some embodiments,” or “some aspects,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments or aspects.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The above summary of the present invention is not intended to describe each disclosed embodiment or every implementation of the present invention. The description that follows more particularly exemplifies illustrative aspects. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

Illustrative embodiments of the present invention are described in detail below with reference to the attached drawing figures, and wherein:

FIG. 1 shows a study flow diagram for the study described in Example 1.

FIG. 2 shows Monocyte Distribution Width, White Blood Cell Count, and Neutrophil-to-Lymphocyte Ratio comparisons by group. The dashed line represents cutoff value of respective test.

FIG. 3A shows Lactate and FIG. 3B shows C-reactive protein by group compared to leukocyte measures for the same sub-populations. The dashed line in each panel represents cutoff value of respective test. Sepsis and septic shock are defined according to Sepsis-3 consensus definition. Abbreviations: CRP: c-reactive protein, MDW:

FIG. 4 shows Monocyte Distribution Width, White Blood Cell Count, and Neutrophil-to-Lymphocyte Ratio comparisons by group in the immunosuppressed patient sub-group. The dashed line in each panel represents cutoff value of respective test. The immunosuppressed patient sub-group were identified by having neutropenia or an active problem meeting criteria for an immunocompromised state.

FIG. 5 is a schematic depiction of an example operating environment, in accordance with aspects of the present disclosure.

FIG. 6 is schematic depiction of an example analyzer, in accordance with aspects of the present disclosure.

FIG. 7 is a schematic depiction of an example analyzer process, in accordance with aspects of the present disclosure.

FIG. 8 is a schematic depiction of an example analysis engine, in accordance with aspects of the present disclosure.

FIG. 9 depicts a flow chart of an example method for evaluating sepsis or septic shock from a blood sample, in accordance with aspects of the present disclosure.

FIG. 10 depicts an example transformation of blood sample parameters to an index that identifies a patient on a spectrum of low- to high-risk for sepsis and septic shock, in accordance with aspects of the present disclosure.

FIG. 11 depicts an example process and laboratory output for a clinician to visualize, in accordance with aspects of the present disclosure.

### DETAILED DESCRIPTION

This disclosure describes methods of and systems for screening for sepsis or septic shock in a patient and methods of ruling out sepsis or septic shock in a patient using white blood cell count (WBC), a monocyte cell population parameter, or neutrophil-to-lymphocyte ratio (NLR), or a combination thereof, in the blood sample from the patient. In some aspects, the method may preferably be used for screening patients in the emergency department (ED).

Current tools for ED-based sepsis screening are limited. Clinical scoring systems that rely on routinely available information are advantageous due to their universal applicability. The original systemic inflammatory response syndrome (SIRS) criteria remain widely used for sepsis screening but lack specificity; patients with acute noninfectious illness often screen positive. (Sprung et al. Intensive Care Med. 2006; 32 (3): 421-427; Bone et al. Chest. 1992; 101 (6): 1644-1655.) SIRS criteria were excluded from the third iteration of consensus sepsis definitions (SEP-3), which introduced the quick sequential organ failure assessment score (qSOFA) and has been applied for sepsis screening and prognostication. (Singer et al. JAMA. 2016; 315 (8): 801-810.) Unfortunately, qSOFA has proven to lack sensitivity in the ED where patients often present prior to manifesting overt signs of organ failure. (Serafim et al. Chest. 2018; 153 (3): 646-655.) Biomarkers may serve as adjuncts for sepsis screening and diagnosis. However, currently available biomarkers, including lactate, c-reactive protein and procalcitonin, may perform sub-optimally due to limited diagnostic accuracy, detectable signal delay and/or lack of widespread availability in the clinical setting. (Al Jalbout et al. The Journal of Applied Laboratory Medicine. 2019; 3 (4): 724-729; Pierrakos et al. Crit Care. 2020; 24 (1): 287.)

While work to leverage automated algorithms to support sepsis care in EDs is growing, a single simple biomarker that could enable early identification in an undifferentiated population would be highly valuable. To date, none has been discovered. Lactate, CRP and procalcitonin are commonly employed for sepsis risk-stratification, but none exhibit optimal diagnostic performance when used in isolation. (Lippi, Clinical Chemistry and Laboratory Medicine (CCLM). 2019; 57 (9): 1281-1283.) Lactate, the only biomarker whose use is recommended by consensus guidelines, is a nonspecific marker of cellular dysfunction and its elevation does not occur until late in the disease course; its use is recommended for prognostication and monitoring response to therapy rather than case identification. (Rhodes et al. Intensive Care Medicine. 2017; 43 (3): 304-377; Casserly et al. Critical Care Medicine. 2015; 43 (3).) CRP has been shown to lack sensitivity and specificity for sepsis in undifferentiated populations and to be particularly unreliable in the ED setting. (Wasserman et al. Medicine (Baltimore). 2019; 98 (2); Wu et al. Ann Intensive Care. 2017; 7.) Procalcitonin may have a role in the differentiation of bacterial from viral infections, but recent data suggest its sensitivity for invasive infection is unacceptably low to warrant its use as a screening tool. (Goodlet et al. Open Forum Infect Dis. 2020; 7 (4); Gregoriano et al. J Thorac Dis. 2020; 12 (Suppl 1): S5-$15.) Utility of these biomarkers for early sepsis screening is further undermined by availability that depends on clinical suspicion and is subject to practice variability. For example, lactate, CRP, and procalcitonin are not used clinically in all EDs.

Complete blood cell count (CBC), a test that counts the cells that make up blood, is the most commonly ordered laboratory panel worldwide (Horton et al. Am J Clin Pathol. 2019; 151 (5): 446-451) and a CBC is ordered for most patients arriving in an emergency department. Although CBC has been suggested as a source of a considerable amount of useful information in a patient suffering from sepsis, typical practice is to overlook most of this data, focusing only on a single component, white blood cell count (WBC). (Farkas, J Thorac Dis. 2020; 12 (Suppl 1): S16-S21.) Little support is available to practicing clinicians on using CBC results in aggregate for sepsis detection.

WBC was incorporated into original consensus criteria for sepsis but has been excluded from more recent sepsis definitions due to its poor diagnostic accuracy when used in isolation. (Bone et al. Chest. 1992; 101 (6): 1644-1655.) Other components of the CBC, including neutrophil count, lymphocyte count, and neutrophil-to-lymphocyte ratio (NLR), have been suggested as possible markers for sepsis (Ljungström et al. PLOS One. 2017; 12 (7): e0181704; Martins et al. Rev Bras Ter Intensiva. 2019; 31 (1): 64-70) but are limited in their ability to correlate to patient groups that are more severely ill. Traditional screening tools such as systemic inflammatory response syndrome (SIRS) criteria, National Early Warning Score (NEWS), Modified Early Warning Score (MEWS), quick Sequential Organ Failure Assessment (qSOFA), and Sequential Organ Failure Assessment (SOFA) criteria also exhibit poor diagnostic accuracy. Islam et al. Comput Methods Programs Biomed 2019; 170:1-9

When CBC is obtained from a blood sample, using an analyzer such as a hematology analyzer on the same blood sample may further provide data about a subpopulation of cells that is much richer than simply a count or proportion of those cells compared to other subpopulations of cells within a sample. For example, a monocyte cell population parameter that reflects monocyte activation may be obtained. One such monocyte parameter is monocyte distribution width (referred to herein as MDW, and also referred to as monocyte anisocytosis). MDW represents the volume distribution of the monocyte population in a blood sample; thus, this morphometric parameter reflects variability in monocyte cell volume.

Morphological changes in monocyte cell volume occur early as a result of pathogen recognition-induced monocyte activation, and thus MDW is altered early in disease trajectory. MDW has demonstrated capability in identification of patients with sepsis in high-risk populations. (Crouser et al. Crit Care Med. 2019; 47 (8): 1018-1025; Crouser et al. Chest. 2017; 152 (3): 518-526; Crouser et al. Intensive Care. 2020; 8:33.) Furthermore, MDW and WBC in combination have been proposed for evaluation of sepsis status. (See, for example, PCT Application No. PCT/US2019/028486.)

This disclosure describes that when MDW is considered with WBC and NLR, as further described herein (including, for example, in Example 1), surprisingly good predictability for both sepsis and septic shock are achieved. For example, the AUC for both sepsis and septic shock reached 0.86 (Table 2) and a sensitivity of 92.2% for sepsis and 97.7% for septic shock was achieved. Moreover, the results suggest that MDW has an important additive role with WBC and NLR, with the combination of the three parameters providing substantially better discrimination of patient outcomes than that of WBC, NLR, or MDW alone or any two of these parameters in combination. The finding that the combination of three distinct parameters from a single laboratory panel (the CBC differential) could achieve such high diagnostic performance was unexpected. The CBC has been used in routine practice, including as a screening laboratory test for infections, for decades. However, use of this combination of CBC parameters has never been described and no study had previously reported this level of discrimination for sepsis and septic shock using any combination of CBC parameters. Also surprising, the sensitivities achieved using the combination of parameters was multiplicative rather than additive.

### Methods of Screening for Sepsis or Septic Shock

In some aspects, this disclosure describes methods of screening for sepsis or septic shock in a patient. The method includes calculating neutrophil-to-lymphocyte ratio (NLR) for a blood sample from the patient; characterizing white blood cell count (WBC) for the blood sample; and calculating a monocyte cell population parameter for the blood sample. The method further includes comparing WBC, the monocyte cell population parameter, and NLR to a threshold value or a threshold range. For example, NLR may be compared to a first predetermined threshold value, WBC to a predetermined threshold range, and the monocyte cell population parameter to a second predetermined threshold value. At least one of these comparisons is used to determine if the patient has an increased risk of sepsis or septic shock. In some aspects, the method is an in vitro method.

In some aspects, screening for sepsis or septic shock includes diagnosing sepsis or detecting sepsis. Additionally or alternatively, however, screening for sepsis or septic shock may include predicting development of sepsis or septic shock within 6 hours, 12 hours, or 24 hours.

In some aspects, determining whether the patient has an increased risk of sepsis or septic shock includes determining whether at least one of WBC, the monocyte cell population parameter, and NLR is greater than a predetermined threshold value or outside of a predetermined threshold range. For example, the method may include determining NLR is greater than the first predetermined threshold value, WBC is outside of the predetermined threshold range, or the monocyte cell population parameter is greater than the second predetermined threshold value, or a combination thereof. In some aspects, determining whether the patient has an increased risk of sepsis or septic shock includes determining NLR is greater than a first predetermined threshold value and further determining the monocyte cell population parameter is greater than a predetermined threshold value or WBC is outside of a predetermined threshold range, or both. In some aspects, determining whether the patient has an increased risk of sepsis or septic shock includes determining NLR is greater than a first predetermined threshold value and further determining the monocyte cell population parameter is greater than a predetermined threshold value.

In some aspects, determining whether the patient has an increased risk of sepsis or septic shock comprises determining NLR is greater than a first predetermined threshold value, WBC is outside of a predetermined threshold range, and the monocyte cell population parameter is greater than a second predetermined threshold value.

Each of the first predetermined threshold value, the second predetermined threshold value, and the predetermined threshold range may be selected by a person having skill in this area including, for example, a clinician. Normal range values are known in the art and may vary slightly depending on the lab (due, for example, to the method of testing or the processing of specimens). In some aspects, the threshold value may include multiple thresholds. In some aspects, a threshold range may include multiple ranges. For example, for each parameter, different thresholds may be provided for sepsis versus septic shock.

In some aspects, the monocyte cell population parameter reflects monocyte activation. For example, the monocyte cell population parameter may preferably include monocyte distribution width (MDW). When the monocyte cell population parameter includes MDW, the second predetermined threshold value may be 19, 20, 21, 22, 23, or 24. In such aspects, determining whether the patient has an increased risk of sepsis or septic shock comprises determining MDW is greater than the second predetermined threshold value. For example, the second predetermined threshold value may be 20, and determining whether the patient has an increased risk of sepsis or septic shock may include determining MDW is greater than 20.

In some aspects, the lower end of the threshold range for WBC is 3.4×10⁹ cells/L, 4×10⁹ cells/L, 4.5×10⁹ cells/L, or 5×10⁹ cells/L. In some aspects, the upper end of the threshold range for WBC is 9.6×10⁹ cells/L, 10×10⁹ cells/L, 11×10⁹ cells/L, or 12×10⁹ cells/L. Exemplary threshold ranges may include, for example, 4×10⁹ cells/L to 12×10⁹ cells/L, 4.5×10⁹ cells/L to 11×10⁹ cells/L, 4×10⁹ cells/L to 11×10⁹ cells/L, 5×10⁹ cells/L to 10×10⁹ cells/L, etc. In such aspects, determining whether the patient has an increased risk of sepsis or septic shock comprises determining WBC is outside of the threshold range. For example, the threshold range for WBC may be 4×10⁹ cells/L to 12×10⁹ cells/L, and determining whether the patient has an increased risk of sepsis or septic shock may include determining WBC is less than 4×10⁹ cells/L or greater than 12×10⁹ cells/L.

In some aspects, the first predetermined threshold value is 6, 7, 8, 9, 10, 11, or 12. In such aspects, determining whether the patient has an increased risk of sepsis or septic shock may include determining NLR is greater than the first predetermined threshold value. For example, the first predetermined threshold value may be 10, and determining whether the patient has an increased risk of sepsis or septic shock may include determining NLR is greater 10.

In some aspects, the method is an automated method. When the method is an automated method, the method may include determining whether the patient has an increased risk of sepsis or septic shock using a data processing module. The data processing module may include a processor and a tangible non-transitory computer readable medium. The computer readable medium may be programmed with any suitable computer application. For example, in some aspects, the computer readable medium is programmed with a computer application that, when executed by the processor, causes the processor to compare NLR to a first predetermined threshold value to provide a first comparison, to compare the monocyte cell population parameter to a second predetermined threshold value to provide a second comparison, to compare WBC to a third predetermined threshold value and a fourth predetermined threshold value to provide a third comparison, and to determine the risk of sepsis or septic shock based on the first comparison, the second comparison, and the third comparison. In such aspects, determining the risk of sepsis or septic shock indicates suspicion of sepsis or septic shock if the first comparison is higher than the first predetermined threshold value, the second comparison is higher than the second predetermined threshold value, and the third comparison is either lower than the third predetermined threshold value or higher than the fourth predetermined threshold value. In some aspects, the first predetermined threshold value (that is, the threshold value for NLR) is 10. In some aspects, the monocyte cell population parameter includes the standard deviation of monocyte volume, for example, monocyte distribution width (MDW), and the second predetermined threshold value (that is, the threshold value for MDW) is 20. In some aspects, the third predetermined threshold value is 12×10⁹ cells/L and the fourth predetermined threshold value is 4×10⁹ cells/L (that is, the threshold range for WBC is 4×10⁹ cells/L to 12×10⁹ cells/L). In some aspects, the automated method may further include indicating in a sample test report suspicion of a sepsis or septic shock. In some aspects, the automated method may include delivering a hydrodynamically focused stream of the blood sample toward a cell interrogation zone of an optical element; and measuring, with an electrode assembly, current (DC) impedance of cells of the blood sample passing individually through the cell interrogation zone; wherein the module determines the standard deviation of monocyte volume based on the DC impedance measurement of cells of the blood sample.

In some aspects, the patient may present with a non-specific complaint. In some aspects, the patient may present with a symptom of a systemic inflammatory condition.

In some aspects, the patient may preferably present to an emergency department.

In some aspects, the method further includes administering a treatment for sepsis. Exemplary treatments for sepsis include administering an antibiotic preparation, an intravenous fluid, a vasopressor, a corticosteroid, insulin, an analgesic, a sedative, or an immune enhancing therapy, or a combination thereof.

In some aspects, the method may further include determining if a treatment for sepsis has been administered or determining the success of the treatment for sepsis.

### Methods of Ruling Out Sepsis or Septic Shock

In some aspects, this disclosure describes methods of ruling out sepsis or septic shock in a patient. The method includes calculating neutrophil-to-lymphocyte ratio (NLR) for a blood sample from the patient; characterizing white blood cell count (WBC) for the blood sample; and calculating a monocyte cell population parameter for the blood sample. The method further includes comparing WBC, the monocyte cell population parameter, and NLR to a threshold value or a threshold range. For example, NLR may be compared to a first predetermined threshold value, WBC to a predetermined threshold range, and the monocyte cell population parameter to a second predetermined threshold value. At least one of these comparisons is used for determining whether the patient has an increased risk of sepsis or septic shock. In some aspects, the method is an in vitro method.

In some aspects, determining whether the patient is not at risk of sepsis or septic shock comprises determining whether at least one of WBC, the monocyte cell population parameter, and NLR is less than a predetermined threshold value or inside of a predetermined threshold range. In some aspects, determining whether the patient is not at risk of sepsis or septic shock comprises determining NLR is less than a first predetermined threshold value and further determining WBC is inside of a predetermined threshold range, or the monocyte cell population parameter is less than a second predetermined threshold value, or both. In some aspects, determining whether the patient is not at risk of sepsis or septic shock comprises determining NLR is less than a first predetermined threshold value and further determining the monocyte cell population parameter is less than a second predetermined threshold value.

In some aspects, determining whether the patient is not at risk of sepsis or septic shock comprises determining NLR is less than a first predetermined threshold value, WBC is inside of a predetermined threshold range, and the monocyte cell population parameter is less than a second predetermined threshold value.

Each of the first predetermined threshold value, the second predetermined threshold value, and the predetermined threshold range may be selected by a person having skill in this area including, for example, a clinician. Normal range values are known in the art and may vary slightly depending on the lab (due, for example, to the method of testing or the processing of specimens). In some aspects, the threshold value may include multiple thresholds. In some aspects, a threshold range may include multiple ranges.

In some aspects, the monocyte cell population parameter reflects monocyte activation. For example, the monocyte cell population parameter may include a monocyte volume measurement. In such aspects, determining whether the patient is not at risk of sepsis or septic shock comprises determining the monocyte cell population parameter is less than the second predetermined threshold value. In one example, the monocyte cell population parameter may preferably include monocyte distribution width (MDW). When the monocyte cell population parameter includes MDW, the second predetermined threshold value may be 19, 20, 21, 22, 23, or 24. In such aspects, determining whether the patient is not at risk of sepsis or septic shock includes determining MDW is less than the second predetermined threshold value. For example, the second predetermined threshold value may be 20, and determining whether the patient is not at risk of sepsis or septic shock comprises determining MDW is less than 20.

In some aspects, the lower end of the threshold range for WBC is 3.4×10⁹ cells/L, 4×10⁹ cells/L, 4.5×10⁹ cells/L, or 5×10⁹ cells/L. In some aspects, the upper end of the threshold range for WBC is 9.6×10⁹ cells/L, 10×10⁹ cells/L, 11×10⁹ cells/L, or 12×10⁹ cells/L. Exemplary threshold ranges may include, for example, 4×10⁹ cells/L to 12×10⁹ cells/L, 4.5×10⁹ cells/L to 11×10⁹ cells/L, 4×10⁹ cells/L to 11×10⁹ cells/L, 5×10⁹ cells/L to 10×10⁹ cells/L, etc. In such aspects, determining whether the patient is not at risk of sepsis or septic shock includes determining WBC is inside of the threshold range. For example, the threshold range for WBC may be 4×10⁹ cells/L to 12×10⁹ cells/L, and determining whether the patient is not at risk of sepsis or septic shock may include determining WBC is greater than 4×10⁹ cells/L and less than 12×10⁹ cells/L.

In some aspects, the first predetermined threshold value is 6, 7, 8, 9, 10, 11, or 12. In such aspects, determining whether the patient is not at risk of sepsis or septic shock comprises determining NLR is less than the first predetermined threshold value. For example, the first predetermined threshold value may be 10, and determining whether the patient is not at risk of sepsis or septic shock comprises determining NLR is less than 10.

In some aspects, the patient may present with a non-specific complaint. In some aspects, the patient may present with a symptom of a systemic inflammatory condition.

In some aspects, the patient may preferably present to an emergency department.

### Operating Environment and Exemplary Analyzers

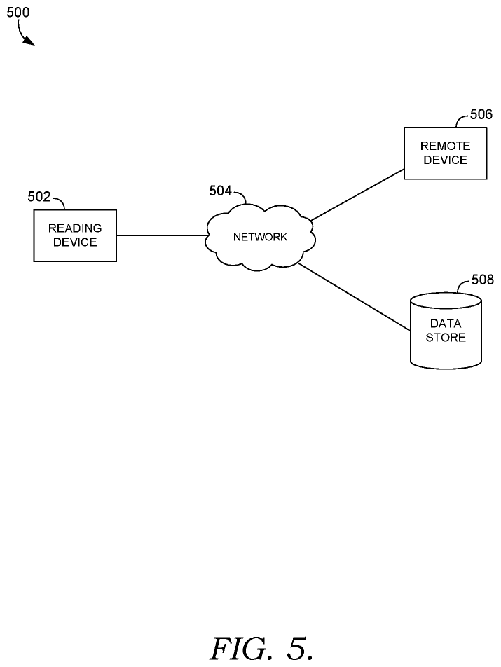

Turning to FIG. 5, an example operating environment 500 is depicted in accordance with some aspects described herein. Generally, example operating environment 500 includes systems that can facilitate diagnostic, predictive, and medical intervention action described herein. Operating environment 500 is one example of a suitable environment and system architecture for implementing an embodiment of the disclosure. Some embodiments may be implemented as a system, comprising one or more computers and associated network and equipment, upon which a method or computer software application is executed. Accordingly, aspects of the present disclosure may take the form of an embodiment combining software and hardware aspects that may all generally be referred to herein as a “module” or “system.” Further, the methods of the present disclosure may take the form of a computer application embodied in computer readable media having machine-readable application software embodied thereon. In this regard, a machine-readable storage media may be any tangible medium that can contain or store a software application for use by the computing apparatus.

Some aspects of example operating environment 500 include at least one analyzer 502. An analyzer is a clinical diagnostic machine capable of measuring one or more anatomical or physiological properties of a sample, including: vitals, metabolic measurements (also referred to as blood chemistry); cell counts; viral protein, viral gene or microbial cell measurements; urine measurements; genomic characterizations; or mass spectrometry and/or immunological measurements. Analyzers as used herein include workcells or modular systems where two or more types of measurements are taken; for example, a workcell comprising blood chemistry and immunoassay. In some aspects, the analyzer may include a hematology analyzer.

Some aspects of example operating environment 500 include network 504. Network 504 generally facilitates communication between analyzer 502 and any other device communicatively coupled to network 504. As such, network 504 can include access points, routers, switches, or any other network component commonly understood to facilitate communication among devices. By way of example network 504 can include one or more wide area networks, one or more local area networks, one or more public networks, one or more private networks, one or more telecommunications networks, or any combination thereof. In other words, network 504 may include multiple networks, or a network of networks, but is depicted in a simple form so as not to obscure aspects described herein.

Some aspects of example operating environment 500 include remote device 506. Remote device 506 can take on a variety of forms, such as a personal computer (PC), a smart phone, a smart watch, a laptop computer, a mobile phone, a mobile device, a tablet computer, a wearable computer, a personal digital assistant (PDA), any combination of these delineated devices, or any other device that can communicate directly or indirectly with an analyzer (e.g., analyzer 502) and/or a data store (for example, data store 508). For example, in a particular aspect remote device 506 comprises a work station PC that can execute a local client application. The local client application can communicatively connect with analyzer 502, data store 508, or both. For example, the local client application can be an application that facilitates user interaction with the analyzer 502. The local client application. In another example, the local client application can be an electronic medical record system application that facilitates user interaction with an electronic medical record system maintained by a data store.

Some aspects of example operating environment 500 includes one or more data stores 508. Data store 508 generally stores, maintains, and communicates data through network 504. Data store 508 may comprise hardware, software, firmware in any combination. For example, data store 508 can include an electronic medical record (EMR) system. The EMR system can store medical information (for example, demographic, physical, biological, and so forth) about a plurality of individuals. In other words, an EMR is a real-time, comprehensive collection of patent data including medical history, physician notes, diagnoses, medication, allergies, immunizations, laboratory test results and vital signs. An EMR system stores and maintains a plurality of EMRs.

In another example, data store 508 may comprise a laboratory information system (LIS). A LIS is a software system that stores, processes, and manages laboratory analyzer data, and information about an individual, including sample measurements. Laboratory test results derived from an individual's biological sample, such as WBC and MDW, may also be input to the LIS manually, by a laboratory professional, indirectly through laboratory middleware connected to one or more analyzers, or directly from an analyzer. In some aspects, an LIS system can add or modify patent data stored in an EMR system.

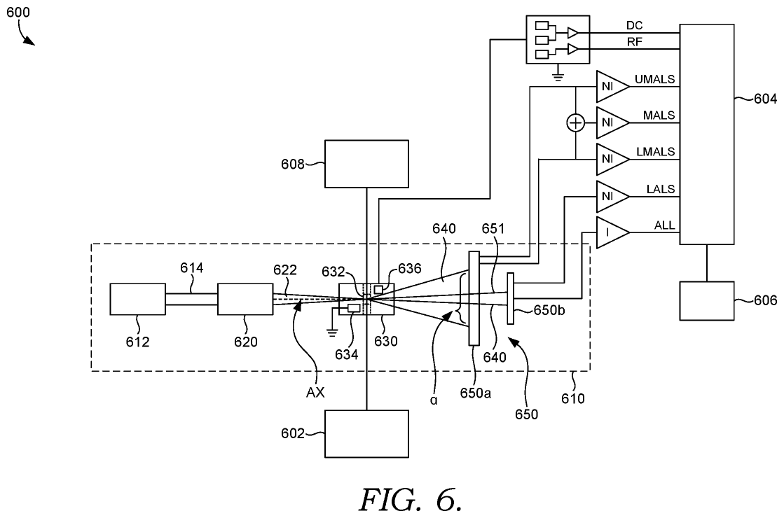

Turning to FIG. 6, a depiction of an example analyzer 600 is provided consistent with aspects described herein. Analyzer 600 depicts components in a system which may be used to take measurements of a sample, for example a blood sample. As will be understood by those skilled in the art, analyzers are available that work on many principles, including electrical impedance, stained fluorescence analysis, cell image analysis, and light scatter analysis. In particular, many commercially available hematology analyzers use a combination of these methodologies. For example, a Beckman Coulter DxH™ 900 Hematology Analyzer uses electrical impedance (also called DC current) to size and count cells and uses Radio Frequency (RF), light loss, and light scatter to evaluate cell morphology and further distinguish sub-populations of cells. Exemplary systems and methods are described, for example, in U.S. Pat. No. 5,125,737. As will be appreciated from the disclosure of U.S. Pat. No. 5,125,737, there is often more than one way of distinguishing cells in a blood sample. For example, cells may be distinguished based on volume (often measured by impedance), or by light scatter, or by combinations of parameters. If distinguished by light scatter, different angles of light scatter may be used, such as low angle light scatter (LALS), axial light loss (ALL), upper median angle light scatter (UMALS), and the like. In some cases, cells that are similar in size and morphology may best be distinguished using combinations of different measures, which may use plots (for example, with one measure on the x-axis and another measure on the y-axis) or formulas, such as ratios or sums. As one example, eosinophils have several light scatter measures similar to neutrophils, and can be difficult to distinguish based on any single measurement. However, by looking at medium angle light scatter (MALS), a combination of UMALS and lower median angle light scatter (LMALS), eosinophils can be clearly distinguished from neutrophils as well as monocytes, lymphocytes, and basophils.

The D×H systems use high-speed, high-resolution analog-to-digital conversion with Digital Signal Processing (DSP) circuitry to measure multiple parameters for each cellular event. DSP algorithms analyze the cellular data digitally, providing cellular definition and resolution. Differential accuracy and flagging technology are obtained by combining the additional light scatter measurements with data analysis techniques to further define and separate cell populations. For example, in some cases, MALS may be transformed to a modified rotated MALS (RMALS) parameter through a mathematical transformation to eliminate overlap and create distinct neutrophil, lymphocyte, monocyte and eosinophil populations, enabling optimal analysis and visual confidence in the results. Current internal and visual differential data transformations include: RMALS (rotated MALS), opacity (conductivity minus the size aspect), SOP (stretched opacity), and non-linear AL2 transformations. The D×H systems report “VCS parameters”.

Other hematology analyzers, such as those manufactured by Abbott, Sysmex, and Mindray, use the cluster of differentiation (CD) system to differentiate white blood cell populations. CD molecules can act in numerous ways, often acting as receptors or ligands; by which a signal cascade is initiated, altering the behavior of the cell. Some CD proteins do not play a role in cell signaling, but have other functions, such as cell adhesion. The CD system nomenclature commonly used to identify cell markers thus allows cells to be defined based on what molecules are present on their surface. There are more than 350 CD molecules identified for humans. For example, monocytes can be identified with CD45+ and CD14+. Using fluorescently labeled antibodies, these cell markers can used to sort cells. Fluorescence activated cell sorting (FACS) provides a method of sorting a heterogeneous mixture of cells into two or more containers, a single cell at a time, based upon the specific light scattering and fluorescent characteristics of each cell. Fluorescence flow cytometry or FACS may also provide information about cell composition of a labeled cell. For example, information about cell density or complexity may be obtained by measuring light scattered by the cell and information about cell size and internal structure may be obtained by measuring the fluorescence signal intensity of the cell. Systems based on fluorescence flow cytometry or FACS report “FACS parameters.”

There are many thousands of possible combinations of sensor readings and calculated relationships that might correlate to a particular characteristic of a blood sample, and, once subpopulations of cells have been identified, a particular subpopulation of cells may be further characterized by one or more sensor readings (such as, for example, LALS, ALL, UMALS, LMALS, MALS, impedance, etc.), in addition to or in lieu of cytochemical staining, marker affinity, or other cell identification techniques. That is, hematology analyzers can often provide data about a subpopulation of cells that is much richer than simply a count or proportion of those cells compared to other subpopulations of cells within a sample. One example is Monocyte Distribution Width (MDW), a calculation of the standard deviation of cell volumes within the subpopulation of monocytes within a blood sample. This characterization of the monocyte population is associated with sepsis, as described, for example, in PCT Application Nos. PCT/US2017/014708, PCT/US2020/041535, PCT/US2019/28486, PCT/US2020/041541, PCT/US2020/41548, and PCT/US2019/028487. MDW may be determined by passing an electric current through a blood sample and measuring the volume of individual cells passing through a measurement module based on measuring the amplitude of the resulting impedance measurement (e.g., in a flow cell 630 of a system such as shown in FIG. 6). This volume may also be measured by a system which transmits light through a blood sample and measures the resulting light scatter to determine cell volume. In some cases, more than one characterization of a subpopulation of cells or relationship between subpopulation of cells may be indicative of the same or related conditions, such as viral infection, sepsis, anemia, leukemia, etc.

As shown in FIG. 6, analyzer 600 includes a transducer module 610 having a light or irradiation source such as a laser 612 emitting a beam 614. The laser 612 can be, for example, a 635 nm, 5 mW, solid-state laser. In some instances, analyzer 600 may include a focus-alignment system 620 that adjusts beam 614 such that a resulting beam 622 is focused and positioned at a cell interrogation zone 632 of a flow cell 630. In some instances, the flow cell 630 receives a sample aliquot from a preparation system 602. Various fluidic mechanisms and techniques can be employed for hydrodynamic focusing of the sample aliquot within flow cell 630.

In some instances, the aliquot generally flows through the cell interrogation zone 632 such that its constituents pass through the cell interrogation zone 632 one at a time. In some cases, an analyzer 600 may include a cell interrogation zone or other feature of a transducer module or blood analysis instrument such as those described in U.S. Pat. Nos. 5,125,737; 6,228,652; 7,390,662; 8,094,299; 8,189,187; and 9,939,453, the contents of which are incorporated herein by reference for all purposes. For example, a cell interrogation zone 632 may be defined by a square transverse cross-section measuring approximately 50×50 microns, and having a length (measured in the direction of flow) of approximately 65 microns. Flow cell 630 may include an electrode assembly having first and second electrodes 634, 636 for performing DC impedance and/or RF conductivity measurements of the cells passing through cell interrogation zone 632. Signals from electrodes 634, 636 can be transmitted to the analysis system 604. The electrode assembly can analyze volume and conductivity characteristics of the cells using low-frequency current and high-frequency current, respectively. For example, low-frequency DC impedance measurements can be used to analyze the volume of each individual cell passing through the cell interrogation zone. High-frequency RF current measurements can be used to determine the conductivity of cells passing through the cell interrogation zone. Because cell walls act as conductors to high frequency current, the high frequency current can be used to detect differences in the insulating properties of the cell components, as the current passes through the cell walls and through each cell interior. High frequency current can be used to characterize nuclear and granular constituents and the chemical composition of the cell interior.

The light source in FIG. 6 has been described as a laser, however, the light source may alternatively or additionally include a xenon lamp, an LED lamp, an incandescent lamp, or any other suitable source of light, including combinations of the same or different kinds of lamps (e.g., multiple LED lamps or at least one LED lamp and at least one xenon lamp). As shown in FIG. 6, for example, incoming beam 622 irradiates the cells passing through cell interrogation zone 632, resulting in light propagation within an angular range a (e.g. scatter, transmission) emanating from the zone 632. Exemplary systems are equipped with sensor assemblies that can detect light within one, two, three, four, five, or more angular ranges within the angular range a, including light associated with an extinction or axial light loss measure. As shown, light propagation 640 can be detected by a light detection assembly 650, optionally having a light scatter detector unit 650A and a light scatter and/or transmission detector unit 650B. In some instances, light scatter detector unit 650A includes a photoactive region or sensor zone for detecting and measuring upper median angle light scatter (UMALS), for example, light that is scattered or otherwise propagated at angles relative to a light beam axis within a range from 20 to 42 degrees. In some instances, UMALS corresponds to light propagated within an angular range from between 20 to 43 degrees, relative to the incoming beam axis, which irradiates cells flowing through the interrogation zone. Light scatter detector unit 650A may also include a photoactive region or sensor zone for detecting and measuring lower median angle light scatter (LMALS), for example, light that is scattered or otherwise propagated at angles relative to a light beam axis within a range from 10 to 20 degrees. In some instances, LMALS corresponds to light propagated within an angular range from between 9 to 19 degrees, relative to the incoming beam axis which irradiates cells flowing through the interrogation zone.

A combination of UMALS and LMALS is defined as median angle light scatter (MALS), which may be light scatter or propagation at angles between 9 degrees and 43 degrees relative to the incoming beam axis which irradiates cells flowing through the interrogation zone. One of skill in the art will understand that these angles (and the other angles described herein) may vary somewhat based on the configuration of the interrogation, sensing and analysis systems.

As shown in FIG. 6, the light scatter detector unit 650A may include an opening 651 that allows low angle light scatter or propagation 640 to pass beyond light scatter detector unit 650A and thereby reach and be detected by light scatter and transmission detector unit 650B. According to some embodiments, light scatter and transmission detector unit 650B may include a photoactive region or sensor zone for detecting and measuring lower angle light scatter (LALS), for example, light that is scattered or propagated at angles relative to an irradiating light beam axis of less than 5.1 degrees. In some instances, LALS corresponds to light propagated at an angle of less than 9 degrees, relative to the incoming beam axis, which irradiates cells flowing through the interrogation zone. In some instances, LALS corresponds to light propagated at an angle of less than 10 degrees, relative to the incoming beam axis, which irradiates cells flowing through the interrogation zone. In some instances, LALS corresponds to light propagated at an angle of 1.9 degrees±0.5 degrees, relative to the incoming beam axis, which irradiates cells flowing through the interrogation zone. In some instances, LALS corresponds to light propagated at an angle of 3.0 degrees±0.5 degrees, relative to the incoming beam axis, which irradiates cells flowing through the interrogation zone. In some instances, LALS corresponds to light propagated at an angle of 3.7 degrees±0.5 degrees, relative to the incoming beam axis, which irradiates cells flowing through the interrogation zone. In some instances, LALS corresponds to light propagated at an angle of 5.1 degrees±0.5 degrees, relative to the incoming beam axis, which irradiates cells flowing through the interrogation zone. In some instances, LALS corresponds to light propagated at an angle of 7.0 degrees±0.5 degrees, relative to the incoming beam axis, which irradiates cells flowing through the interrogation zone. In each instance, LALS may correspond to light propagated an angle of 1.0 degrees or more. That is, LALs may correspond to light propagated at angles between 1.0 degrees and 1.9 degrees; between 1.0 degrees and 3.0 degrees; between 1.0 degrees and 3.7 degrees; between 1.0 degrees and 5.1 degrees, between 1.0 degrees and 7.0 degrees, between 1.0 degrees and 9.0 degrees; or between 1.0 degrees and 10.0 degrees.

According to some embodiments, light scatter and transmission detector unit 650B may include a photoactive region or sensor zone for detecting and measuring light transmitted axially through the cells, or propagated from the irradiated cells, at an angle of 0 degrees relative to the incoming light beam axis. In some cases, the photoactive region or sensor zone may detect and measure light propagated axially from cells at angles of less than 1 degree relative to the incoming light beam axis. In some cases, the photoactive region or sensor zone may detect and measure light propagated axially from cells at angles of less than 0.5 degrees relative to the incoming light beam axis less. Such axially transmitted or propagated light measurements correspond to axial light loss (ALL or AL2). As noted in previously incorporated U.S. Pat. No. 7,390,662, when light interacts with a particle, some of the incident light changes direction through the scattering process (i.e., light scatter) and part of the light is absorbed by the particles. Both of these processes remove energy from the incident beam. When viewed along the incident axis of the beam, the light loss can be referred to as forward extinction or axial light loss. Additional aspects of axial light loss measurement techniques are described in U.S. Pat. No. 7,390,662 at column 5, line 58 to column 6, line 4.

As such, the analyzer 600 provides means for obtaining light propagation measurements, including light scatter and/or light transmission, for light emanating from the irradiated cells of the biological sample at any of a variety of angles or within any of a variety of angular ranges, including ALL and multiple distinct light scatter or propagation angles. For example, light detection assembly 650, including appropriate circuitry and/or processing units, provides a means for detecting and measuring UMALS, LMALS, LALS, MALS, and ALL.

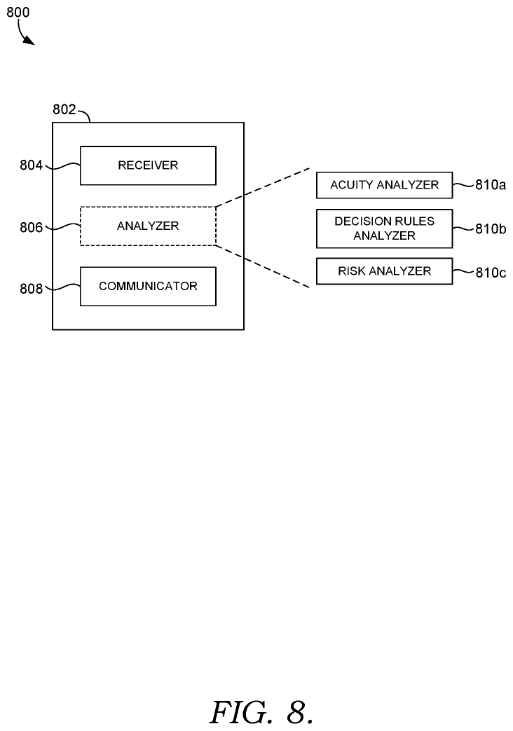

Wires or other transmission or connectivity mechanisms can transmit signals from the electrode assembly (e.g. electrodes 634, 636), light scatter detector unit 650A, and/or light scatter and transmission detector unit 650B to the analysis system 604 for processing. For example, measured DC impedance, RF conductivity, light transmission, and/or light scatter parameters can be provided or transmitted to the analysis system 604 for data processing. In some instances, analysis system 604 may include computer processing features and/or one or more modules or components, which can evaluate the measured parameters, identify and enumerate biological sample constituents, and correlate a subset of data characterizing elements of the biological sample with one or more features or parameters of interest. Some aspects of analysis system 604 include an analysis engine such as described in relation to FIG. 8.

Additionally, or alternatively, as depicted in FIG. 6, analyzer 600 may generate or output a report 606 presenting measurements made or parameters calculated for the sample. The measurements made or parameters calculated for a sample can include UMALS, LMALS, LALS, MALS, ALL, WBC, MDW, monocyte %, absolute lymphocyte count (ALC), lymphocyte %, eosinophil %, absolute neutrophil count (ANC), neutrophil %, or any combination thereof. As further described herein, it may be particularly advantageous in some aspects for the parameters to include MDW, WBC, absolute lymphocyte count (ALC), absolute neutrophil count (ANC), and for the absolute lymphocyte count (ALC) and absolute neutrophil count (ANC) to be used to calculate a neutrophil-to-lymphocyte ratio.

In some instances, excess biological sample from transducer module 610 can be directed to an external (or alternatively internal) waste system 608. In some instances, the analyzer 600 may include one or more features of a transducer module or blood analysis instrument such as those described in previously incorporated U.S. Pat. Nos. 5,125,737; 6,228,652; 8,094,299; 8,189,187; and 9,939,453.

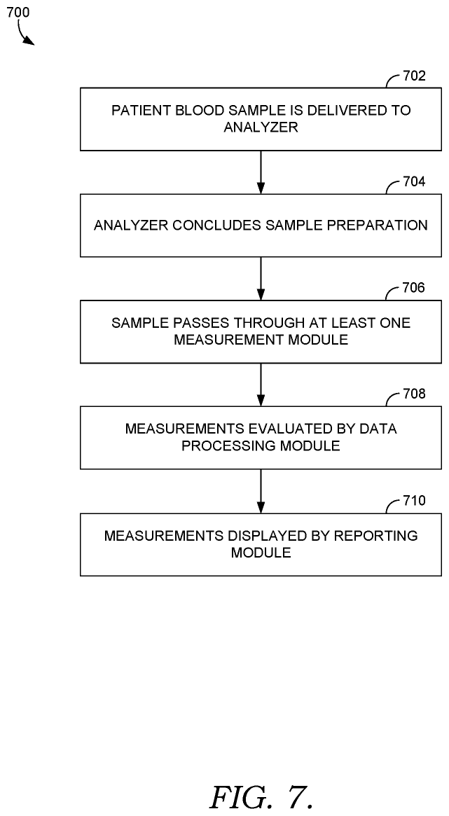

FIG. 7 schematically depicts an exemplary analyzer process 700, for example, which can optionally utilize the analyzer 600 of FIG. 6. In this embodiment, at the step 702, an individual's blood sample may be delivered to the analyzer, at which point the analyzer may prepare the sample for analysis. Once the sample preparation is concluded at step 704, the sample may pass through one or more measurement modules at the step 706. The measurement modules of the step 706 can include a conductivity module, a light scatter module, an RF module, or any combination thereof. Other modules may be used instead of or in addition to a conductivity module or a light scatter module. For example, a hematology analyzer may use sensors to detect dyes or fluorescent markers, imaging, immunoassay markers, size sorting, or other approaches to identify cells or other sample components. Sample measurements may then be evaluated by a data processing module in the step 708. In some aspects, once the sample measurements are complete, the measurements may be displayed by a reporting module in the step 710. Additionally, or alternatively, once the sample measurements are complete the measurements may be communicated to an analysis engine for further processing, such as the example analysis engine 800 of FIG. 8.

FIG. 8 depicts an example analysis engine 800, in accordance with aspects described herein. Aspects of analysis engine 800 can be incorporated into a processing feature and/or module or component of an analyzer (such as analysis system 604 depicted in FIG. 6), an application executed by a remote device (e.g., remote device 506 depicted in FIG. 5), or can operate as an independent component of an operating environment (e.g., operating environment 500 depicted in FIG. 5).

Generally, the analysis engine 800 evaluates a set of measurements or parameters, identifies and enumerates biological sample constituents, and correlates a subset of data characterizing elements of the biological sample with one or more features or parameters of interest. As such, analysis engine 800 includes a receiver module 804, an analyzer module 806, and a communicator module 808.

A receiver, such as receiver 802, generally collects measurements made or parameters calculated based on analysis of an individual's sample. The data (for example, measurements made or parameters calculated) can be received directly from a subsystem of an analyzer or from a data store in some aspects. Receiver 802 can use any data collection technique known in the art.

Data analyzer 806 includes modules that include logical expressions for the evaluation of measurements and parameters received by the analysis engine 800. The logical expressions can include linear or parallel processes that evaluate measurements made by or parameters calculated by a hematology analyzer, such as analyzer 502 described in relation to FIG. 5 or analyzer 600 described in relation to FIG. 6. The data analyzer 806 includes at least one of an acuity analyzer 810a, a decision rules analyzer 810b, and a risk analyzer 810c.

Acuity analyzer 810a comprises a library of rules, models, and logic expressions, in any combination that facilitate the determination of a probability and/or risk of one or more outcomes based on one or more parameters or characteristics of a blood sample. A potential outcome can be associated with a recommendation, treatment, or intervention in some aspects. For example, where an individual's outcome corresponds with a risk of shock, a recommendation to transfer the individual to an intensive care/critical care unit can be associated therewith.

Decision rules analyzer 810b comprises a library of decision rules. A decision rule is a logic expression that compares an individual parameter or characteristic of a blood sample with a threshold value. The decision rules analyzer 810b assembles one or more decision rules from the library to build a logical expression that the analysis engine can evaluate. In one aspect, the analyzer 810b can utilize a linear combination or two or more parameters. In combination, the decision rules can be used to determine a probability that an individual associated with a blood sample currently has a condition, such as, for example, an infection, including viral infection.

Risk analyzer 810c can include rules, models, logic expressions, in any combination that are configured to forecast medical conditions. For example, the risk analyzer module can characterize the information received from an analyzer to determine an individual's risk of developing sepsis. Additionally, some aspects of the risk analyzer module can character the information received from the analyzer to determine a probability of sepsis severity.

In some aspects, the data analysis engine 800 can incorporate the operations of one or more analyzer modules to generate an output. For example, the decision rules maintained by a decision rules analyzer 810b can be used to determine if an individual currently has a condition, such as an infection. In response to determination that the individual has a probability of infection above a particular threshold, some aspects of the data analysis engine 800 can activate the risk analyzer 810c to facilitate the determining if the individual is at an elevated risk of developing sepsis or septic shock. In response to the determination that the individual has an elevated risk of developing sepsis or septic shock, some aspects of the data analysis engine 800 can activate the acuity analyzer 810a to facilitate determining a recommended level of care or disposition. In alternative aspects, the acuity analyzer 810a may first identify an individual is at risk of needing critical care and/or at risk of in-hospital mortality, e.g., within 48 hours of obtaining the blood sample, then one or more of the decision rules analyzer 810b and/or the risk analyzer 810c can be utilized for further determinations.

Communicator 808 generally communicates the results of the analysis engine 800 to at least one predetermined target. In some aspects, the predetermined target can include a remote device that is executing a local client of a laboratory information system or a local client of an electronic medical record system (e.g., remote device 506 described in relation to FIG. 5). In such aspects, the results can include presentation of a visual display or audio signal that provides a recommendation of care, recommendation to authorize discharge, a recommendation of diagnosis, or an alert that the individual corresponding to the analyzed sample may develop sepsis or other severe condition.

In some aspects, the predetermined target can include a data store maintaining a laboratory information system or an electronic medical record system (for example, data store 508 described in relation to FIG. 5). In such aspects, the communicated results can include entering orders for the individual associated with the analyzed sample's medical records. For example, the orders can include transferring the individual to a critical care unit, increasing monitoring of the individual by medical personal or devices, or specific testing or standard of care protocols.

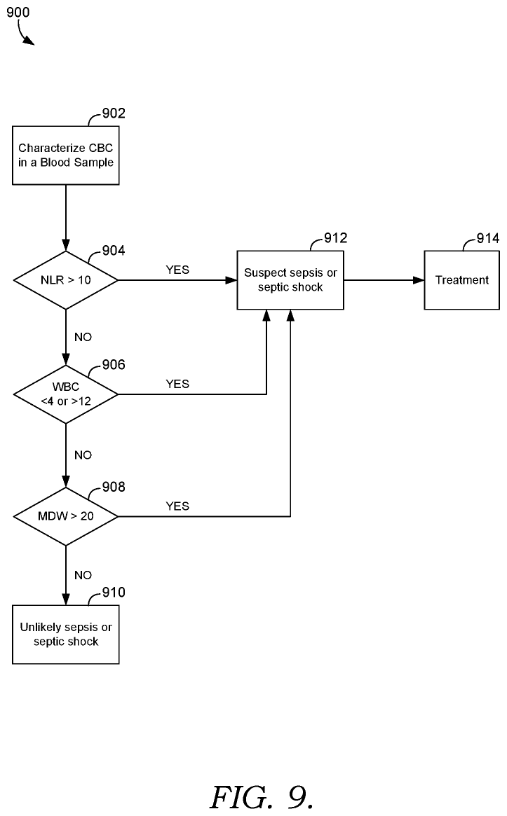

As described in more detail above, data analysis engine 800 includes at least one analyzer that processes the measurements or parameters provided to the analysis engine. The processing can include rules, models, logic expressions, in any combination that are configured to detect and/or forecast medical conditions. For example, some aspects of a decision rules analyzer (for example, decision rules analyzer 810b described in relation to FIG. 8) can include programs that characterize the information received from an analyzer to determine a probability that an individual has developed sepsis or is likely to develop sepsis. In particular, an exemplary method 900 for assessing sepsis or septic shock is depicted in FIG. 9 in accordance with some aspects described herein. Method 900 may generally be described as a “decision rules” approach, where individual parameters or characteristics of a blood sample are considered against threshold values for each parameter or characteristic.

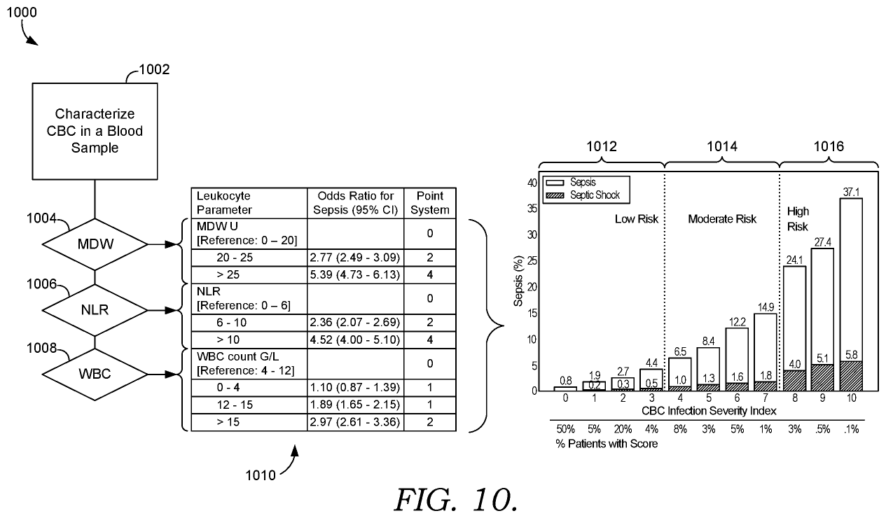

As described below in relation to FIG. 10, additional or alternative embodiments of a method executed by a data analysis engine (e.g., data analysis engine 800) includes a weighted scoring method for the parameters or characteristics of a blood sample.

### Hematological Assessment of Sepsis and Septic Shock

As described above, an analyzer may count and differentiate the various cells included in a blood sample. Some aspects of a method 900 include characterizing blood cells as part of a complete blood count (CBC) in a blood sample at block 902. The CBC characterization may include characterizing a variety of different parameters. In one example, in a particular aspect of block 902, method 900 includes determining the number (or count) of white blood cells present per liter of blood. In another example, in a particular aspect of block 902, method 900 includes determining a percentage of WBCs that are lymphocytes or an absolute number of white blood cells that are lymphocytes. In a further example, in a particular aspect of block 902, method 900 includes determining a percentage of WBCs that are neutrophils or an absolute number of WBCs that are neutrophils. In yet another example, in a particular aspect of block 902, method 900 includes determining a monocyte parameter including, for example, a monocyte distribution width (MDW). Based on the information collected, an analyzer can determine, for example, the white blood cell count (WBC), lymphocyte percentage, neutrophil percentage, lymphocyte count, neutrophil count, neutrophil-to-lymphocyte ratio (NLR), or monocyte distribution width (MDW), or any combination thereof. For example, at block 902, the method may further include determining, by the analyzer, the absolute neutrophil count and the absolute lymphocyte count and use these values to determine the NLR. In an alternative aspect of block 902, an analyzer engine may query an individual's medical record for a data value that corresponds to the most recent values for white blood cell count (WBC), lymphocyte percentage, neutrophil percentage, lymphocyte count, neutrophil count, neutrophil-to-lymphocyte ratio (NLR), or monocyte distribution width (MDW), or a combination thereof.

At decision block 904, the analyzer further determines whether the NLR determined in block 902 is greater than a first predetermined threshold value (including, for example, 6, 7, 8, 9, 10, 11, or 12). In some aspects, if NLR is greater than the first predetermined threshold value (for example, 10, as shown in FIG. 9), the sample test report may indicate suspicion of a sepsis or septic shock shown as block 912.

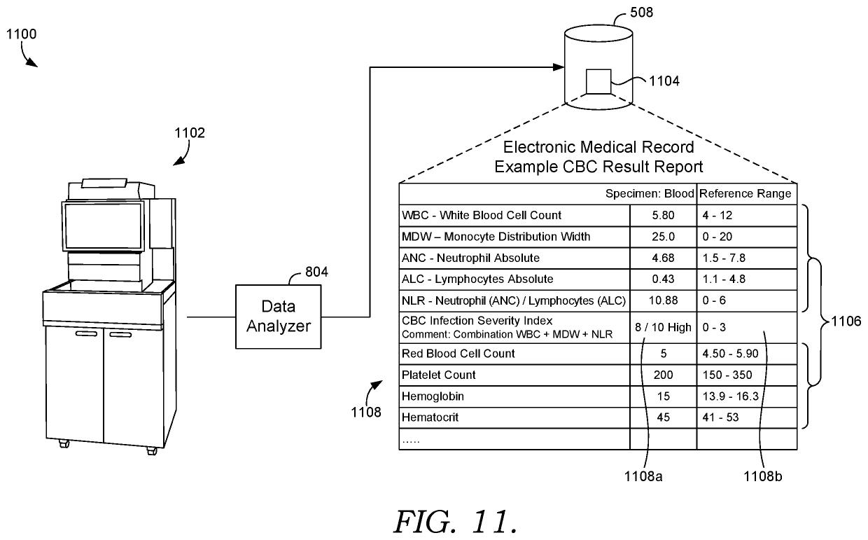

Additionally, or alternatively (if it is determined that NLR is less than the first predetermined threshold value), the method may proceed to decision block 906. Decision block 906 includes determining if the number of white blood cells per liter in the patient's blood is in a threshold range. The lower end of the threshold range for WBC may be, for example, 3.4×10⁹ cells/L, 4×10⁹ cells/L, 4.5×10⁹ cells/L, or 5×10⁹ cells/L; the upper end of the threshold range may be 9.6×10⁹ cells/L, 10×10⁹ cells/L, 11×10⁹ cells/L, or 12×10⁹ cells/L. Exemplary threshold ranges may include, for example, 4×10⁹ cells/L to 12×10⁹ cells/L, 4.5×10⁹ cells/L to 11×10⁹ cells/L, 4×10⁹ cells/L to 11×10⁹ cells/L, 5×10⁹ cells/L to 10×10⁹ cells/L, etc. In some aspects, if the white blood cell count (WBC) is less than the lower end of the threshold range (for example, 4×10⁹ cells/L, as shown in FIG. 9) or greater than the upper end of the threshold range (for example, 12×10⁹ cells/L, as shown in FIG. 9), the sample test report (such as is described in FIG. 11) may indicate suspicion of a sepsis or septic shock shown as block 912.

Additionally, or alternatively (if it is determined that NLR is less than the first predetermined threshold value and WBC is outside of the threshold range), the method may proceed to decision block 908. At decision block 908, the method includes determining whether monocyte distribution width (MDW) value is greater than a second predetermined threshold value (including, for example, 19, 20, 21, 22, 23, or 24) in the blood sample. In other words, the analyzer determines whether the standard deviation of the distribution of the monocyte volumes, as reported by a MDW value determined in block 902 is greater than the second predetermined threshold value in the monocytes counted by the analyzer. In some aspects, if MDW is greater than the second predetermined threshold value (for example, 20, as shown in FIG. 9), the sample test report may indicate suspicion of a sepsis or septic shock shown as block 912.

If it is determined that NLR is greater than the first predetermined threshold value; WBC is outside of the threshold range; and MDW is less than the second predetermined threshold value, the method 900 may proceed to block 910. At block 910, the analyzer may determine that sepsis or septic shock is unlikely.

Additionally or alternatively, although not illustrated in FIG. 9, a sample test report may indicate suspicion of a sepsis or septic shock (shown as block 912 in FIG. 9) if more than one (for example, 2 or more parameters) are greater than the predetermined threshold value or threshold range.

At block 912, an analyzer may generate a suspect message. In some instances, a suspect message may include a flag, message, or other signal on a test report to indicate possible sepsis or septic shock to a clinician or researcher. In some aspects, the suspect message may include an audio or visual message communicated to a remote device that indicates that the individual associated with the sample has a possible viral infection. The indication may be provided on a screen, such as a display for a hematology analyzer, Laboratory Information System (LIS) or Electronic Medical Record (EMR), or may be provided in a print-out, fax, e-mail or other digital or hard copy report of the hematology test results.

In some aspects, a suspect sepsis or septic shock message may prompt additional testing of the patient, including, for example, an additional standard of care sepsis test.

In some aspects, a suspect sepsis or septic shock message may prompt treatment 914 of the individual adapted to possible sepsis or septic shock. In some aspects, a suspect sepsis or septic shock message may prompt the clinician user that a patient is at risk for sepsis or septic shock. Exemplary treatments for sepsis or septic shock may include administering an antibiotic preparation, an intravenous fluid, a vasopressor, a corticosteroid, insulin, an analgesic, a sedative, or an immune enhancing therapy, or a combination thereof.

As further described here, it might be advantageous for the analyzer to further include other clinical data. Such clinical data could be incorporated through the use of the same analyzer or an addition analyzer. Incorporation of other clinical data may further include the use of an algorithm. Exemplary additional clinical data may include patient demographics, medical history, presenting complaint and vital signs, etc. In some aspects, the clinical data may preferably be clinical data available to the clinician and/or the analyzer prior to CBC results.

Additional or alternative embodiments of a method executed by a data analysis engine (e.g., data analysis engine 800) may include a weighted scoring method for the parameters or characteristics of a blood sample. For example, a data analysis engine may execute operations or processes to perform one or more steps of method 1000 shown in FIG. 10. Similar to method 900, aspects of method 1000 include characterizing blood cells as part of a complete blood count (CBC) in a blood sample at block 1002. The CBC characterization may include characterizing a variety of different parameters. In one example, in a particular aspect of block 1002, method 1000 includes determining the number (or count) of white blood cells present per liter of blood. Alternatively or additionally, in a particular aspect of block 1002, method 1000 includes determining a percentage of WBCs that are lymphocytes or an absolute number of white blood cells that are lymphocytes. In a further example, in a particular aspect of block 1002, method 1000 includes determining a percentage of WBCs that are neutrophils or an absolute number of WBCs that are neutrophils. In yet another example, in a particular aspect of block 1002, method 1000 includes determining a monocyte parameter including, for example, a monocyte distribution width (MDW). Based on the information collected, an analyzer can determine, for example, the white blood cell count (WBC), lymphocyte percentage, neutrophil percentage, lymphocyte count, neutrophil count, neutrophil-to-lymphocyte ratio (NLR), or monocyte distribution width (MDW), or any combination thereof. For example, at block 1002, the method may further include determining, by the analyzer, the absolute neutrophil count and the absolute lymphocyte count and use these values to determine the NLR. In an alternative aspect of block 1002, an analyzer engine may query an individual's medical record for a data value that corresponds to the most recent values for white blood cell count (WBC), lymphocyte percentage, neutrophil percentage, lymphocyte count, neutrophil count, neutrophil-to-lymphocyte ratio (NLR), or monocyte distribution width (MDW), or a combination thereof.

In a particular embodiment of method 1000, the data analysis engine discretizes one or more of the values (or counts) determined during the characterization of the blood sample. For example, at block 1004 MDW is discretized, at block 1006 NLR is discretized, and at block 1008 WBC is discretized. For example, the discretization of WBC or any other parameter may be a truncation, rounding, or other approximation of a measured value to a discrete count using, for example, Euler-Maruyama method, the zero-order hold, or any other suitable method. As depicted in FIG. 10, each discretized value is analyzed with a set of decision rules 1010 associated with each of the discretized parameters. Decision rules 1010 depict example parameter ranges for determination of MDW values, NLR values, and WBC values. For example, MDW values comprised between 0 and 20 are scored by the weighted scoring decision rules of a data analysis engine as a 0 (zero), values greater than 20 and less than or equal to 25 are scored as a 2, and values greater than 25 are scored as a 4. For another example, NLR values between 0 and 6 are scored by the weighted scoring decision rules of a data analysis engine as a 0 (zero), values greater than 6 and less than or equal to 10 are scored as a 2, and values greater than 10 are scored as a 4. For another example, WBC values between 0 and 4 are scored by the weighted scoring decision rules of a data analysis engine as a 1, values greater than 4 and less than or equal to 12 are scored as a 0, values greater than 12 and less than or equal to 15 are scored as a 1, and values greater than 15 are scored as a 2. Although other weighted decision rules are contemplated, this discretization was determined based on a univariate assessment of the relationship between the parameter and sepsis outcome (e.g., descriptive statistics and graphical plots) of multiple observational studies. Logistic regression analyses was performed with sepsis (sepsis-3 criteria) as the dependent variable and discretized leukocyte parameters as independent variables. The odds ratios for each of discrete category for the leukocyte parameters were identified and the weights for a point system were defined based on the odds ratio. For example, the objective is to translate these odds ratios (weights) to a simple scoring system between 0 and a set maximum. In the depicted example the maximum points was set at a value of 10. Accordingly, the set of odds ratios were summed (e.g., 2.77+5.39+2.36+4.52+1.10+1.89+2.97=12.9) and scaled to the desired maximum (e.g., 10/12.9) to get a translation factor of 0.78. Each odds ratio is multiplied by the translation factor (0.78) and rounded to the nearest whole number to produce index points depicted in decision rules 1010.

In some embodiments of method 1000, the analyzer (e.g., data analysis engine 800) classifies the risk associated with the blood sample based on the decision rules. The risk classification includes a plurality of potential classes based on the computed sum of the index points for each of the analyzed parameters. As depicted in FIG. 10, a low risk class 1012 may include patients with blood samples with a cumulative score less than or equal to 3; a moderate risk class 1014 may include patients with blood samples with a cumulative score greater than 3 and less than or equal to 7; and, a high risk class 1016 may include patients with blood samples with a cumulative score greater than 7.

Similar to method 900, method 1000 includes generating a suspect message. In some instances, a suspect message may include a flag, message, or other signal on a test report to indicate possible sepsis or septic shock to a clinician or researcher. In some aspects, the suspect message may include an audio or visual message communicated to a remote device that indicates that the individual associated with the sample has a possible viral infection. The indication may be provided on a screen, such as a display for a hematology analyzer, Laboratory Information System (LIS) or Electronic Medical Record (EMR), or may be provided in a print-out, fax, e-mail or other digital or hard copy report of the hematology test results.

In some aspects, a suspect sepsis or septic shock message may prompt additional testing of the patient, including, for example, an additional standard of care sepsis test.

In some aspects, a suspect sepsis or septic shock message may prompt treatment of the individual adapted to possible sepsis or septic shock. In some aspects, a suspect sepsis or septic shock message may prompt the clinician user that a patient is at risk for sepsis or septic shock. Exemplary treatments for sepsis or septic shock may include administering an antibiotic preparation, an intravenous fluid, a vasopressor, a corticosteroid, insulin, an analgesic, a sedative, or an immune enhancing therapy, or a combination thereof.

### Analyzers and Algorithms

As described above, an analysis engine may include a risk analyzer configured to process the measurements or parameters provided to the analysis engine. The risk analyzer can include rules, models, logic expressions, in any combination that are configured to detect and/or forecast medical conditions. The data analyzer (e.g., data analyzer 806 of FIG. 8) may include a machine learning algorithm, including, for example, a decision tree-based classifier, a simple logical classifier, a neural network, a logistic regression algorithm, or the like. The processing may be used to assist in screening for sepsis or septic shock in a patient or to rule out sepsis or septic shock in a patient. Additionally, or alternatively, a machine learning algorithm may be used to compute the parameters and rules for a weighted scoring method (e.g., as described in reference to method 1000 of FIG. 10).

In some aspects, the machine learning algorithm processes an input to provide output data. The input may include WBC, the monocyte cell population parameter, and/or NLR (or whether WBC, the monocyte cell population parameter, and/or NLR exceed a threshold value or a threshold range), and the output may include information indicative of whether the patient has an increased risk of sepsis or septic shock. In some aspects, an input may include all of WBC, the monocyte cell population parameter, and NLR. In some aspects, an input may include information regarding whether any of WBC, the monocyte cell population parameter, and NLR exceed a threshold value or a threshold range. The output data may include a numerical value that specifies the risk of sepsis or septic shock. The output data may further include a rationale for the numerical value that specifies the risk of sepsis or septic shock. The rationale may include, for example, a text-based message that identifies the risk of sepsis associate with the patient based on the output data and includes contextual information regarding the relative severity of the risk (e.g., “low”, “low risk”, “moderate”, “moderate risk”, “high, or “high risk”) based on the predefined risk classification (e.g., as obtained by carrying out method 1000 as described above and depicted in FIG. 10).

In some aspects, the output data may be subject to an additional transformation, described, for example, by a fixed function, to provide an index score. In some aspects, the index score may specify the probability that the patient has an increased risk of sepsis or septic shock. Alternatively, the index score may specify the probability that the patient does not have an increased risk of sepsis or septic shock. In some aspects, the index score may be the result of a weighted scoring method. In some aspects, the index score may be the result of comparing a parameter (for example, the monocyte cell population parameter and/or NLR) to more than one threshold value or of comparing a parameter (for example, WBC) to more than one threshold range.

Rapid simultaneous interpretation of WBC, NLR and MDW would be challenging in a fast-paced ED environment, and processing in the at least one analyzer may provide a more immediate interpretation to a clinical care provide. Further, it is likely that additional precision could be achieved through algorithms that incorporate other clinical data such as patient demographics, medical history, presenting complaint, and vital signs; all available prior to CBC results. (Levin et al. Ann Emerg Med. 2018; 71 (5): 565-574.e2.)

For example, some aspects of an acuity analyzer (for example, acuity analyzer 810a described in relation to FIG. 8) can include programs that characterize the information received from an analyzer to determine an acuity for individual. In some aspects, an acuity analyzer can include programs that characterize the information received from an analyzer to: identify individuals for discharge and/or evaluate whether or not an individual is responding to care. In certain aspects, an acuity analyzer can include programs that characterize the information received from an analyzer to: assess a severity of infection, and/or determine if an individual is at risk of sepsis or shock. In some aspects, an acuity analyzer can include programs that characterize the information received from an analyzer to: stratify the risk associated with febrile neonates, assess or predict a risk of systemic infection, assess or predict if the individual is at risk of exhibiting a hyper-inflammatory state, discern if an individual suspected of an infection has a viral infection, discern if an individual suspected of an infection has a bacterial infection, discern whether antibiotic treatment should be provided or not, discern if an individual is experiencing respiratory exacerbations, for example, cystic fibrosis, related to inflammation and/or infection, assessing a severity of infection of an immune-compromised individual, or a combination thereof.

In certain aspects, systems and methods are provided that are directed to evaluating acuity of an individual. In certain systems, a majority of individuals that arrive at an emergency department (ED) have an uncertain projected clinical course. For instance, in certain scenarios, the majority of individuals entering an ED are likely stable, and the ED will need multiple types of resources, for example, lab tests and/or imaging, to investigate or treat the individual. In certain instances, such individuals with an uncertain projected clinical course can be classified initially as an emergency severity index level 3. The time required to investigate individuals with an uncertain projected clinical course leads to prolonged wait times and dwell times and can be a source of ED or care inefficiencies. Furthermore, diagnosing individuals seeking care in an ED can be challenging due to overlapping symptoms for various ailments, including conditions associated with an infection or not. Prolonged wait times and prolonged times to diagnosis may result in adverse outcomes.

The systems and methods disclosed herein can alleviate one or more of the above problems. For instance, in certain aspects, the systems and methods disclosed herein can assess acuity of an individual. In such aspects, evaluating or assessing acuity of an individual may identify individuals early on that may need increased care, for example, individuals having sepsis or septic shock or having a risk or elevated risk of sepsis or septic shock. These individuals may then be diverted to the appropriate care units, such as a critical care facility (for example, an Intensive Care Unit (ICU)), more efficiently. In the same or alternative aspects, by identifying individuals having a risk of sepsis or severe sepsis early on, via the methods disclosed herein, additional standard of care sepsis tests can then be ordered.

In various aspects, the systems and methods disclosed herein can identify individuals for discharge. For instance, in certain aspects, MDW values (alone or in combination with WBC and/or NLR) may be compared to one or more predetermined criteria to identify an individual as a candidate for discharge. In various aspects, parameter values may be obtained on multiple blood samples over the course of care, or observation. In such aspects, identifying an individual for discharge can aid in freeing up hospital resources, and/or allocate hospital resources more efficiently.

For example, as depicted in FIG. 11 and with passing reference to FIGS. 5-10, an analyzer 1102 may process and analyze biological samples (e.g., blood) and generate output data. Analyzer 1102 may be a hematology instrument (e.g., analyzer 600) or any other laboratory device configured to determine measurements or parameters relevant to blood or its constituents. The output data from analyzer 1102 may include UMALS, LMALS, LALS, MALS, ALL, WBC, MDW, monocyte %, ALC, lymphocyte %, eosinophil %, ANC, neutrophil %, neutrophil-to-lymphocyte ratio, or any combination thereof. A data analyzer (e.g., data analyzer 806), incorporated with analyzer 1102 or remotely executed, accesses the output data. The data analyzer may analyze the data (e.g., as described in relation to FIGS. 7-10). For example, the data analyzer 806 may use a weighted scoring method to generate a sepsis risk score based on a set of rules for the parameters and measurements determined by analyzer 1102. A communicator (e.g., communicator 808 of FIG. 8) transmits, amongst other things, the risk score generated by the data analyzer to a data store (e.g., data store 508 of FIG. 5) that is maintaining an electronic medical record 1104 for a patient associated with the biological sample. The risk score may be added to the electronic medical record 1104 in machine understandable format with executable instructions that define the maximum score, reference range, or both.

In a particular embodiment, the executable instructions include conditional instructions that are triggered in response to predetermined actions. For example, a remote device 506 may execute a local or remote application that provides access the electronic medical record 1104 stored by data store 508. In response to the application requesting a complete blood count (CBC) for the patient (e.g., report data 1106), the instructions may trigger the insertion of the risk score identification 1108, the risk score 1108a, and a reference range 1108b.

In another embodiment, the executable instructions trigger an automatic message on a remote device 506 that is executing an application that provides access the electronic medical record 1104 stored by data store 508 in response to the risk score exceeding a predetermined threshold.

### Computer-Implemented Methods

In a first aspect, this disclosure provides a computer-implemented method that includes obtaining, with a computing device, subject value data for the patient. The subject value data includes at least one of a neutrophil-to-lymphocyte ratio (NLR) for a blood sample from the patient, a white blood cell count (WBC) for the blood sample from the patient, and a monocyte cell population parameter for the blood sample from the patient. The computer-implemented method further includes determining, with the computing device, whether the patient has an increased risk of sepsis or septic shock by using the subject value data for the patient.

In a second aspect, this disclosure provides a computer-implemented method that includes obtaining, with a computing device, subject value data for the patient. The subject value data includes at least one of a neutrophil-to-lymphocyte ratio (NLR) for a blood sample from the patient, a white blood cell count (WBC) for the blood sample from the patient, and a monocyte cell population parameter for the blood sample from the patient. The computer-implemented method further includes determining, with the computing device, whether the patient is not at risk of sepsis or septic shock.

In some aspects, the subject value data includes the neutrophil-to-lymphocyte ratio (NLR) for the blood sample from the patient, the white blood cell count (WBC) for the blood sample from the patient, and the monocyte cell population parameter for the blood sample from the patient.

In some aspects, the monocyte cell population parameter preferably includes monocyte distribution width (MDW).

The subject value data for the patient may further include one or more additional parameters including, for example, patient vitals, such as blood pressure of the patient, blood oxygen level of the patient, heartbeat rate of the patient or the like. Alternatively or additionally, the subject value data for the patient may further include one or more of lymphocyte percentage, neutrophil percentage, lymphocyte count, and neutrophil count.

Determining, with the computing device, whether the patient has an increased risk of sepsis or septic shock may be carried out by using at least a trained machine learning algorithm. Determining, with the computing device, whether the patient is not at risk of sepsis or septic shock may carried out by using at least a machine learning algorithm. Accordingly, the methods according to the first aspect and/or the method according to the second aspect of the present disclosure may be at least partly implemented as trained machine learning algorithm or any other artificial intelligence-based algorithm.

The machine learning algorithm may including a trained artificial intelligence-based algorithm. For example, the aforementioned machine learning algorithm may include at least a decision tree, at least a neural network, at least a gradient boosting algorithm, and/or at least a logistic regression algorithm. In some aspects, the machine learning algorithm consists of a decision tree, a neural network, a gradient boosting algorithm, or a logistic regression algorithm.

The obtained subject value data of the patient may be processed by means of the trained classifier of the computing device. For example, the computing device may include a classifier or classifier circuitry, which may include a trained machine learning algorithm, a trained gradient boosting algorithm, or any other AI-based algorithm or circuitry. The classifier may be part of a control circuitry of the computing device or may be implemented as separate classifier circuitry in the computing device.

Exemplarily, determining whether the patient has an increased risk of sepsis includes processing the subject value data for the patient by using the machine learning algorithm. For instance, the obtained subject value data of the patient may be processed by means of the trained classifier of the computing device. According to an embodiment of the second aspect of the present disclosure, determining whether the patient is not at risk of sepsis or septic shock includes processing the subject value data for the patient by using the machine learning algorithm. Hence, in particular, according to the present disclosure, the subject value data of the patient may be the input of the trained machine learning algorithm.

In some embodiments of the first aspect of the present disclosure, determining whether the patient has an increased risk of sepsis or septic shock, includes generating output data. According to some embodiments of the second aspect of the present disclosure, determining whether the patient is not at risk of sepsis or septic shock includes generating output data. In particular, according to the present disclosure, generating output data may be carried out by processing the subject value data for the patient by using the machine learning algorithm. In some embodiments of the first aspect of the present disclosure, the output data includes information indicative of whether the patient has an increased risk of sepsis or septic shock. According to some embodiments of the second aspect of the present disclosure, the output data includes information indicative of whether the patient is not at risk of sepsis or septic shock includes generating output data.

According to the present disclosure, the output data may include a numerical value. For example, said numerical value may be a value between 0 and 1, or between 0 and 10, or comprised in any other normalized range and may specify the risk of sepsis or septic shock, so that bigger values of the numerical value correspond to higher risks of sepsis or septic shock. Alternatively, said numerical value may specify the risk of sepsis or septic shock so that bigger values of the numerical value correspond to lower risks of sepsis or septic shock.

The output data may further include a rationale for the numerical value that specifies the risk of sepsis or septic shock. The rationale may include, for example, a text-based message.

According to the present disclosure, the output data may be subject to an additional transformation, described, for example, by a fixed function, to provide an index score. According to the first aspect of the present disclosure, the index score may specify the probability that the patient has an increased risk of sepsis or septic shock. In some embodiments of the second aspect of the present disclosure, the index score may specify the probability that the patient does not have risk of sepsis or septic shock. In some aspects, the index score may be the result of a weighted scoring method. In some aspects, the index score may be the result of comparing a parameter (for example, the monocyte cell population parameter and/or NLR) to more than one threshold value or of comparing a parameter (for example, WBC) to more than one threshold range.

In particular, the machine learning algorithm may be trained by using a reference dataset indicative of reference subject values associated with one or more reference patients. The reference dataset may comprise a plurality of reference subject value data. For instance, each reference subject value data of the plurality of reference subject value data includes at least one of: a neutrophil-to-lymphocyte ratio (NLR) of a blood sample from a respective reference subject; a white blood cell count (WBC) for the blood sample from the respective reference subject, and a monocyte cell population parameter for the blood sample from the respective reference subject.

In some aspects, each reference subject value data of the plurality of reference subject value data includes the neutrophil-to-lymphocyte ratio (NLR) of the blood sample from the respective reference subject, the white blood cell count (WBC) for the blood sample from the respective reference subject, and the monocyte cell population parameter for the blood sample from the respective reference subject.

The reference dataset may be used, for example, for training and/or used as training dataset of the machine learning algorithm, the artificial intelligence-based algorithm and/or the classifier of the computing device. For instance, the trained machine learning algorithm, AI-based algorithm and/or classifier may comprise a plurality of parameters, the value of said parameters being determined during training by using the reference dataset. For instance, the computing device may be trained by using raw reference data associated with the one or more reference subjects. Additionally, or alternatively, a machine learning algorithm may be used to compute the parameters and rules for a weighted scoring method (e.g., as obtained by carrying out method 1000 as described above and depicted in FIG. 10).

Each reference subject value data of the plurality of reference subject value data may include sepsis information for the reference subject. According to the first aspect of the present disclosure, the sepsis information for the reference subject is indicative of whether the respective reference subject has an increased risk of sepsis or septic shock. In some embodiments of the second aspect of the present disclosure, the reference subject is indicative of whether the respective reference subject is not at risk of sepsis or septic shock comprises generating output data. During training, the sepsis information comprised in at least a portion of the plurality of reference subject value data may be used to evaluate a loss function, the loss function being used, for example minimized, during training of the machine learning algorithm.

According to the present disclosure, the method may include determining and/or deriving at least a part of the reference dataset by using raw reference data associated with one or more reference patients. The raw reference data may include NLR of blood samples from reference subjects WBC for the blood samples from reference subjects, monocyte cell population parameter for blood sample from reference subjects, sepsis information for reference subjects.

According to the present disclosure, “obtaining, with a computing device, data” or “obtaining, with a computing device, subject value data” may include accessing, by the computing device, the data or subject value data. Alternatively, “obtaining, with a computing device, data” or “obtaining, with a computing device, subject value data” or “obtaining, with a computing device, subject value data for the patient” may include generating the data or subject value data that is, creating the data or subject value data based on one or more inputs. In yet another example, “obtaining, by a computing device, data” or “obtaining, with a computing device, subject value data for the patient” may include accessing a first portion of the data or subject value data and generating a second portion of the data or subject value data from the first portion of the data or subject value data.

In the present disclosure, “accessing, by the computing device, the data” may include retrieving the data, for example, from the at least one memory of said computing device, from the memory of another computing device, or from another remote data storage (a database, a secondary memory, a cloud storage or the like). Accordingly, in some cases, retrieving data may include downloading data. Exemplarily, said computing device may retrieve data in response to a user command and/or to a notification sent by another computing device. Additionally or alternatively, “accessing, by the computing device, the data” may include receiving the data, for example, from a user or another computing device. The two options are not mutually exclusive. For instance, “accessing, by the computing device, the data” may include receiving the data, storing the data in the memory of said computer device, and retrieving the data by accessing said memory.

In the present disclosure, obtaining the subject value data for the patient may include calculating NLR for the blood sample from the patient, characterizing WBC for the blood sample from the patient, and/or calculating the monocyte cell population parameter for the blood sample from the patient. Obtaining the subject value data for the patient may additionally or alternatively, include obtaining data that provides information regarding the relationship between NLR for the blood sample from the patient, WBC for the blood sample from the patient, and/or the monocyte cell population parameter for the blood sample from the patient, and corresponding threshold values or threshold ranges.

The invention is defined in the claims. However, below there is provided a non-exhaustive listing of non-limiting exemplary aspects. Any one or more of the features of these aspects may be combined with any one or more features of another example, embodiment, or aspect described herein.

### EXEMPLARY ASPECTS

Aspect A1 is a method of screening for sepsis or septic shock in a patient, the method comprising: calculating neutrophil-to-lymphocyte ratio (NLR) for the blood sample from the patient;
characterizing white blood cell count (WBC) for a blood sample; calculating a monocyte cell population parameter for the blood sample; comparing NLR to a first predetermined threshold value, WBC to a predetermined threshold range, and the monocyte cell population parameter to a second predetermined threshold value; and determining whether the patient has an increased risk of sepsis or septic shock.
Aspect A2 is the method of Aspect A1, wherein determining whether the patient has an increased risk of sepsis or septic shock comprises determining NLR is greater than the first predetermined threshold value, WBC is outside of the predetermined threshold range, or the monocyte cell population parameter is greater than the second predetermined threshold value, or a combination thereof.
Aspect A3 is the method of Aspect A1 or A2, wherein determining whether the patient has an increased risk of sepsis or septic shock comprises determining NLR is greater than the first predetermined threshold value, WBC is outside of the predetermined threshold range, and the monocyte cell population parameter is greater than the second predetermined threshold value.
Aspect A4 is the method of any of Aspects A1 to A3, wherein the monocyte cell population parameter reflects monocyte activation.
Aspect A5 is the method of any of Aspects A1 to A4, wherein the monocyte cell population parameter comprises monocyte distribution width (MDW).
Aspect A6 is the method of Aspect A5, wherein the second predetermined threshold value is 19, 20, 21, 22, 23, or 24, and wherein determining whether the patient has an increased risk of sepsis or septic shock comprises determining MDW is greater than the second predetermined threshold value.
Aspect A7 is the method of Aspect A6, wherein the second predetermined threshold value is 20, and wherein determining whether the patient has an increased risk of sepsis or septic shock comprises determining MDW is greater than 20.
Aspect A8 is the method of any of Aspects A1 to A7, wherein the lower end of the threshold range for WBC is 3.4×10⁹ cells/L, 4×10⁹ cells/L, 4.5×10⁹ cells/L, or 5×10⁹ cells/L; and the upper end of the threshold range is 9.6×10⁹ cells/L, 10×10⁹ cells/L, 11×10⁹ cells/L, or 12×10⁹ cells/L, and wherein determining whether the patient has an increased risk of sepsis or septic shock comprises determining WBC is outside of the threshold range.
Aspect A9 is the method of Aspect A8, wherein the threshold range is 4×10⁹ cells/L to 12×10⁹ cells/L, and wherein determining whether the patient has an increased risk of sepsis or septic shock comprises determining WBC is less than 4×10⁹ cells/L or greater than 12×10⁹ cells/L.
Aspect A10 is the method of any of Aspects A1 to A7, wherein the first predetermined threshold value is 6, 7, 8, 9, 10, 11, or 12, and wherein determining whether the patient has an increased risk of sepsis or septic shock comprises determining NLR is greater than the first predetermined threshold value.
Aspect A11 is the method of Aspect A10, wherein the first predetermined threshold value is 10, and wherein determining whether the patient has an increased risk of sepsis or septic shock comprises determining NLR is greater than 10.
Aspect A12 is the method of any of Aspects A1 to A11, wherein the patient presents with a non-specific complaint.
Aspect A13 is the method of any of Aspects A1 to A12, wherein the patient presents with a symptom of a systemic inflammatory condition.
Aspect A14 is the method of any of Aspects A1 to A13, wherein the patient present to an emergency department.
Aspect A15 is the method of any of Aspects A1 to A14, wherein the method is an in vitro method.
Aspect A16 is the method of any of Aspects A1 to A15, wherein the method further comprises administering a treatment for sepsis.
Aspect A17 is the method of Aspect A16, where administering a treatment for sepsis comprises administering an antibiotic preparation, an intravenous fluid, a vasopressor, a corticosteroid, insulin, an analgesic, a sedative, or an immune enhancing therapy, or a combination thereof.
Aspect A18 is the method of Aspect A16 or A17, where the method further comprises determining the success of the treatment for sepsis.
Aspect A19 is the method of any of Aspects A1 to A18, wherein the method further comprises discretizing one or more of NLR, WBC, and the monocyte cell population parameter to provide a discretized value for one or more of NLR, WBC, and the monocyte cell population parameter; analyzing the discretized value for one or more of NLR, WBC, and the monocyte cell population parameter with a set of decision rules; defining weights for a points system based on an odds ratio; and producing index points that may be summed to provide an index score.
Aspect B1 is a method of ruling out sepsis or septic shock in a patient, the method comprising: calculating neutrophil-to-lymphocyte ratio (NLR) for the blood sample from the patient; characterizing white blood cell count (WBC) for the blood sample; calculating a monocyte cell population parameter for the blood sample; comparing NLR to a first predetermined threshold value, WBC to a predetermined threshold range, and the monocyte cell population parameter to a second predetermined threshold value; and determining whether the patient is not at risk of sepsis or septic shock.
Aspect B2 is the method of Aspect B1, wherein determining whether the patient is not at risk of sepsis or septic shock comprises determining NLR is less than a first predetermined threshold value; and WBC is inside of a predetermined threshold range, or the monocyte cell population parameter is less than the second predetermined threshold value, or both.
Aspect B3 is the method of Aspect B1 or B2, wherein determining whether the patient is not at risk of sepsis or septic shock comprises determining NLR is less than a first predetermined threshold value, WBC is inside of a predetermined threshold range, and the monocyte cell population parameter is less than a second predetermined threshold value.
Aspect B4 is the method of any of Aspects B1 to B3, wherein the monocyte cell population parameter reflects monocyte activation.
Aspect B5 is the method of any of Aspects B1 to B4, wherein the monocyte cell population parameter comprises monocyte distribution width (MDW).
Aspect B6 is the method of Aspect B5, wherein the second predetermined threshold value is 19, 20, 21, 22, 23, or 24, and wherein determining whether the patient is not at risk of sepsis or septic shock comprises determining MDW is less than the second predetermined threshold value.
Aspect B7 is the method of Aspect B6, wherein the second predetermined threshold value is 20, and wherein determining whether the patient is not at risk of sepsis or septic shock comprises determining MDW is less than 20.
Aspect B8 is the method of any of Aspects B1 to B7, wherein the lower end of the threshold range for WBC is 3.4×10⁹ cells/L, 4×10⁹ cells/L, 4.5×10⁹ cells/L, or 5×10⁹ cells/L; and the upper end of the threshold range is 9.6×10⁹ cells/L, 10×10⁹ cells/L, 11×10⁹ cells/L, or 12×10⁹ cells/L, and wherein determining whether the patient is not at risk of sepsis or septic shock comprises determining WBC is inside of the threshold range.
Aspect B9 is the method of Aspect B8, wherein the threshold range for WBC is 4×10⁹ cells/L to 12×10⁹ cells/L, and wherein determining whether the patient is not at risk of sepsis or septic shock comprises determining WBC is greater than 4×10⁹ cells/L and less than 12×10⁹ cells/L.
Aspect B10 is the method of any of Aspects B1 to B9, wherein the first predetermined threshold value is 6, 7, 8, 9, 10, 11, or 12, and wherein determining whether the patient is not at risk of sepsis or septic shock comprises determining NLR is less than the first predetermined threshold value.
Aspect B11 is the method of Aspect B10, wherein the first predetermined threshold value is 10, and wherein determining whether the patient is not at risk of sepsis or septic shock comprises determining NLR is less than 10.
Aspect B12 is the method of any of Aspects B1 to B11, wherein the patient presents with a non-specific complaint.
Aspect B13 is the method of any of Aspects B1 to B12, wherein the patient presents with a symptom of a systemic inflammatory condition.
Aspect B14 is the method of any of Aspects B1 to B13, wherein the patient present to an emergency department.
Aspect B15 is the method of any of Aspects B1 to B14, wherein the method is an in vitro method.
Aspect B16 is the method of Aspect B1, wherein the method further comprises discretizing one or more of NLR, WBC, and the monocyte cell population parameter to provide a discretized value for one or more of NLR, WBC, and the monocyte cell population parameter; analyzing the discretized value for one or more of NLR, WBC, and the monocyte cell population parameter with a set of decision rules; defining weights for a points system based on an odds ratio; and producing index points that may be summed to provide an index score.
Aspect C1 is a method comprising an automated method for screening for sepsis or septic shock in a patient, the method comprising: calculating neutrophil-to-lymphocyte ratio (NLR) for a blood sample from the patient; characterizing white blood cell count (WBC) for the blood sample; calculating a monocyte cell population parameter for the blood sample; and determining whether the patient has an increased risk of sepsis or septic shock using a data processing module, wherein the data processing module comprises a processor and a tangible non-transitory computer readable medium, and the computer readable medium is programmed with a computer application that, when executed by the processor, causes the processor to compare NLR to a first predetermined threshold value to provide a first comparison, to compare the monocyte cell population parameter to a second predetermined threshold value to provide a second comparison, to compare WBC to a third predetermined threshold value and a fourth predetermined threshold value to provide a third comparison, and to determine the risk of sepsis or septic shock based on the first comparison, the second comparison, and the third comparison.
Aspect C2 is the method of Aspect C1, wherein determining the risk of sepsis or septic shock indicates suspicion of sepsis or septic shock if the first comparison is higher than the first predetermined threshold value, the second comparison is higher than the second predetermined threshold value, and the third comparison is either lower than the third predetermined threshold value or higher than the fourth predetermined threshold value.
Aspect C3 is the method of Aspect C1 or C2, wherein the first predetermined threshold value is 6, 7, 8, 9, 10, 11, or 12.
Aspect C4 is the method of Aspect C3, wherein the first predetermined threshold value is 10.
Aspect C5 is the method of any of Aspects C1 to C4, wherein the monocyte cell population parameter comprises the standard deviation of monocyte volume.
Aspect C6 is the method of Aspect C5, wherein the standard deviation of monocyte volume comprises monocyte distribution width (MDW), and wherein the second predetermined threshold value is 19, 20, 21, 22, 23, or 24.
Aspect C7 is the method of Aspect C6, wherein the standard deviation of monocyte volume comprises monocyte distribution width (MDW), and wherein the second predetermined threshold value is 20.
Aspect C8 is the method of any of Aspects C1 to C5, wherein third predetermined threshold value is 3.4×10⁹ cells/L, 4×10⁹ cells/L, 4.5×10⁹ cells/L, or 5×10⁹ cells/L; and the fourth predetermined threshold value is 9.6×10⁹ cells/L, 10×10⁹ cells/L, 11×10⁹ cells/L, or 12×10⁹ cells/L
Aspect C9 is the method of Aspect C8 wherein the third predetermined threshold value is 12×10⁹ cells/L and the fourth predetermined threshold value is 4×10⁹ cells/L.
Aspect C10 is the method of any of Aspects C1 to C9, further comprising altering a test reporting process based on the evaluation of the sepsis status.
Aspect C11 is the method of any of Aspects C1 to C10, further comprising treating the individual from whom the blood sample was obtained if the sepsis status indicates sepsis.
Aspect C12 is the method of any of Aspects C1 to C11, further comprising: delivering a hydrodynamically focused stream of the blood sample toward a cell interrogation zone of an optical element; and measuring, with an electrode assembly, current (DC) impedance of cells of the blood sample passing individually through the cell interrogation zone; wherein the module determines the standard deviation of monocyte volume based on the DC impedance measurement of cells of the blood sample.
Aspect D1 is the method of any of aspects A1 to A17, wherein determining whether the patient has an increased risk of sepsis or septic shock comprises generating output data by processing input data by using a trained machine learning algorithm, wherein the input data comprises the NLR for a blood sample from the patient, the WBC for the blood sample from the patient, and the monocyte cell population parameter for the blood sample from the patient, and wherein the output data comprises information indicative of whether the patient has an increased risk of sepsis or septic shock.
Aspect D2 is the method of any of aspects B1 to B14, wherein determining whether the patient has an increased risk of sepsis or septic shock comprises generating output data by processing input data by using a trained machine learning algorithm, wherein the input data comprises the NLR for a blood sample from the patient, the WBC for the blood sample from the patient, and the monocyte cell population parameter for the blood sample from the patient, and wherein the output data comprises information indicative of whether the patient is not at risk of sepsis or septic shock.
Aspect D3 is the method of Aspect D1 or Aspect D2, wherein the output data comprises a numerical value that specifies the risk of sepsis or septic shock.
Aspect D4 is the method of Aspect D3, wherein the output data further comprises a rationale for the numerical value that specifies the risk of sepsis or septic shock.
Aspect D5 is the method of any of Aspects D1 to D4, wherein the method further comprises discretizing one or more of NLR, WBC, and the monocyte cell population parameter to provide a discretized value for one or more of NLR, WBC, and the monocyte cell population parameter; analyzing the discretized value for one or more of NLR, WBC, and the monocyte cell population parameter with a set of decision rules; defining weights for a points system based on an odds ratio; and producing index points that may be summed to provide the numerical value that specifies the risk of sepsis or septic shock.
Aspect E1 is a computer-implemented method comprising: obtaining, with a computing device, subject value data for the patient, the subject value data including a neutrophil-to-lymphocyte ratio (NLR) for a blood sample from the patient, a white blood cell count (WBC) for the blood sample from the patient, and a monocyte cell population parameter for the blood sample from the patient; and determining, with the computing device, whether the patient has an increased risk of sepsis or septic shock by using the subject value data for the patient.
Aspect E2 is the method according to Aspect E1, wherein determining whether the patient has an increased risk of sepsis is carried out by using at least a machine learning algorithm.
Aspect E3 is the method according to Aspect E2, wherein determining whether the patient has an increased risk of sepsis comprises processing the subject value data for the patient by using the machine learning algorithm.
Aspect E4 is a computer-implemented method comprising: obtaining, with a computing device, subject value data for the patient, the subject value data including a neutrophil-to-lymphocyte ratio (NLR) for a blood sample from the patient, a white blood cell count (WBC) for the blood sample from the patient, and a monocyte cell population parameter for the blood sample from the patient; determining, with the computing device, whether the patient is not at risk of sepsis or septic shock.
Aspect E5 is the method according to Aspect E4, wherein determining whether the patient is not at risk of sepsis or septic shock is carried out by using at least a machine learning algorithm.
Aspect E6 is the method according to Aspect E5, wherein determining whether the patient is not at risk of sepsis or septic shock comprises processing the subject value data for the patient by using the machine learning algorithm.
Aspect E7 is the method according to any one of the Aspect E2, Aspect E3, Aspect E5 and Aspect E6, wherein the machine learning algorithm is trained by using reference dataset indicative of reference subject values associated with one or more reference patients; wherein the reference dataset comprises a plurality of reference subject value data, each reference subject value data of the plurality of reference subject value data including at least one of: a neutrophil-to-lymphocyte ratio (NLR) of a blood sample from a respective reference subject; a white blood cell count (WBC) for the blood sample from the respective reference subject, and a monocyte cell population parameter for the blood sample from the respective reference subject.
Aspect E8 is the method according to any one of Aspect E1 to Aspect E7, wherein obtaining the subject value data for the patient comprises: calculating NLR for the blood sample from the patient, characterizing WBC for the blood sample from the patient, and/or calculating the monocyte cell population parameter for the blood sample from the patient.
Aspect E9 is the method of any of Aspects E1 to E8, wherein the method further comprises discretizing one or more of NLR, WBC, and the monocyte cell population parameters to provide a discretized value for one or more of NLR, WBC, and the monocyte cell population parameter; analyzing the discretized value for one or more of NLR, WBC, and the monocyte cell population parameter with a set of decision rules; defining weights for a points system based on an odds ratio; and producing index points that may be summed to provide the subject value data for the patient.

The present invention is illustrated by the value for one or more of NLR, WBC, and the monocyte cell population parameterparticular examples, materials, amounts, and procedures are to be interpreted broadly in accordance with the scope and spirit of the invention as set forth herein.

### EXAMPLES

### Example 1

This Example shows a CBC differential panel that includes MDW, white blood cell count (WBC), and neutrophil-to-lymphocyte ratio (NLR) demonstrated strong performance characteristics in a broad ED population suggesting pragmatic value as a rapid screen for sepsis and septic shock.

This Example describes a study that evaluated the performance of monocyte distribution width (MDW) alone and in combination with other routine CBC parameters as a screen for sepsis and septic shock in ED patients. A prospective cohort analyses of adult patients with a CBC collected at an urban ED from January 2020 through July 2021. The performance of MDW, white blood cell count (WBC) and neutrophil-to-lymphocyte-ratio (NLR) to detect sepsis and septic shock (Sepsis-3 Criteria) was evaluated using diagnostic performance measures.

7952 ED patients were included in the cohort with 180 meeting criteria for sepsis; 43 with septic shock and 137 without shock. MDW was highest for patients with septic shock (median 24.8 U, IQR 22.0-28.1) and trended downward for patients with sepsis without shock (23.9 U, IQR 20.2 26.8), infection (20.4 U, IQR 18.2-23.3), then controls (18.6 U, IQR 17.1-20.4). In isolation, MDW detected sepsis and septic shock with an AUC of 0.80 (95% CI 0.77-0.84) and 0.85 (95% CI 0.80-0.91), respectively. Optimal performance was achieved in combination with WBC and NLR for detection of sepsis (AUC 0.86, 95% CI 0.83-0.89) and septic shock (0.86, 95% CI 0.80-0.92).

### Materials and Methods

Study Design and Setting. This prospective cohort study was conducted between Jan. 21, 2020 and Jul. 14, 2021 at the Johns Hopkins Hospital ED in Baltimore, MD. The study was approved by the Institutional Review Board (IRB) and follows Standards for Reporting of Diagnostic Accuracy Studies (STARD) guidelines.

Selection of participants. All adult patients (aged 18 and over) who had a CBC collected within 6 hours of ED arrival, as a part of routine clinical care, were eligible for the study. Patients were enrolled consecutively during time-periods when study team members were present. Patients missing a valid MDW (e.g., low sample volume or poor sample quality), patients with MDW sample analyses performed more than 2 hours after blood collection, and patients missing other CBC parameters (WBC, neutrophils, lymphocytes) within 6 hours of arrival were excluded. Repeat ED visits by the same patient during the study period were also excluded.

Measurements. Demographics, clinical data (presenting complaints, co-morbidities, vital signs, laboratory), and hospital utilization data were collected from the electronic health record (EHR) system. Presenting complaints were entered from a picklist at ED triage and co-morbidities were mined by grouping diagnostic codes (ICD-10) for active problems available in the EHR at patient presentation. (World Health Organization. Classification of Diseases (ICD), available online at www.who.int/standards/classifications/classification-of-diseases (last accessed May 26, 2021); Levin et al. Ann Emerg Med. 2018; 71 (5): 565-574.e2; Agency for Healthcare Research in Quality. AHRQ QI ICD-10-CM/PCS Specification Version 7 Patient Safety Indicators Appendices, available online at www.qualityindicators.ahrq.gov/Downloads/Modules/PSI/V70/TechSpecs/PSI_Appendix_I.pdf las last accessed May 26, 2021).) The quick Sequential Organ Failure Assessment (qSOFA) score (range, 0-3 points) was estimated at triage using the first measurement of systolic blood pressure (<100 mm Hg=1 point), respiratory rate (>22 breaths/min=1 point) and altered mental status (1 point) indicated by a presenting complaint related to altered mental status (Levin et al. Ann Emerg Med. 2018; 71 (5): 565-574.e2) or a Glasgow Coma Score (GCS)<15 reported within 6 hours of ED arrival. (Seymour et al. JAMA 2016; 315 (8): 762). Mortality was defined as in hospital mortality or discharge to hospice. Direct admission to an intensive care unit (ICU), inpatient hospitalization, and length-of-stay (ED presentation to physically exiting the hospital) were also reported.

MDW was analyzed on a UniCel DxH900 analyzer (Beckman Coulter, Inc), software version 1.0 in K2 EDTA tubes. A cutoff value of greater than 20 Units was defined as abnormal. (Sprung et al. Intensive Care Med. 2006; 32 (3): 421-427; Bone et al. Chest. 1992; 101 (6): 1644-1655; Singer et al. JAMA. 2016; 315 (8): 801-810.) MDW measurement was performed by a study team member blind to patient clinical information. MDW was not reported in the EHR; clinicians were blinded to MDW values while providing care to patients enrolled. Other CBC parameters (WBC and NLR) were measured on a separate hematology analyzer used for routine clinical practice and were available to treating clinicians. An abnormal WBC was defined as less than 4×10⁹ cells/L or greater than 12×10⁹ cells/L (Bone et al. Chest. 1992; 101 (6): 1644-1655; Levy et al. Intensive Care Medicine. 2003; 29 (4): 530-538) and an abnormal NLR was defined as greater than 10. (Farkas J Thorac Dis. 2020; 12 (Suppl 1): S16-S21.) Lactate (abnormal defined greater than 2.0 mmol/L) (Singer et al. JAMA. 2016; 315 (8): 801-810) and C-reactive protein (CRP) (abnormal defined greater than 10 mg/L) (Laboratory Tests Reference Ranges. Available online at www.abim.org/Media/bfijryql/laboratory-reference380ranges.pdf) measurements were both performed upon request by the treating team and included in analyses as comparators if performed within 6 hours of ED presentation. The immunosuppressed patient sub-group was defined as those having neutropenia (absolute neutrophil count less than or equal to 1.5×10⁹ cells/L measured within 6 hours of ED arrival) or an active problem meeting criteria for an immunocompromised state. (Agency for Healthcare Research in Quality. AHRQ QI ICD-10-CM/PCS Specification Version 7 Patient Safety Indicators Appendices, available online at www.qualityindicators.ahrq.gov/Downloads/Modules/PSI/V70/TechSpecs/PSI_Appendix_I.372 pdf (last accessed May 26, 2021).)

Outcomes. The primary outcome was manifestation of sepsis with or without shock within 12 hours of CBC collection. For analyses, patients were assigned to four mutually exclusive groups based on previously validated criteria: control, infection, sepsis (without shock), or septic shock. (Singer et al. JAMA. 2016; 315 (8): 801-810; Liu et al. J Biomed Inform. 2021; 121:103879.) Patients met criteria for infection if they either (a) started a new antibiotic over a course of at least 4 days (first to last administration day) and had blood culture ordered within 48 hours of ED arrival, or (b) met ICD-10 code diagnostic criteria for an infection. (Liu et al. J Biomed Inform. 2021; 121:103879.) Patients with a shorter course of antibiotics qualified if death occurred before 4 days from treatment initiation. The Sepsis-3 definition using Sequential Organ Failure Assessment (SOFA) (Singer et al. JAMA. 2016; 315 (8): 801-810) was used as reference standard to define the sepsis groups (sepsis without shock and septic shock). Patients were assigned to the sepsis group if they met criteria for infection and met at least one of the following SOFA criteria within 12 hours of CBC collection: (a) vasopressor initiation, (b) initiation of mechanical ventilation, (c) doubling in serum creatinine level, (d) decrease by 50% of estimated glomerular filtration rate relative to baseline (e) bilirubin level greater than 2.0 mg/dL and doubling from baseline (f) platelet count less than 100×10⁹/L and greater than 50% decline from baseline (baseline had to be greater than 100×10⁹/L), or (g) lactate greater than 2.0 mmol/L. Patients meeting sepsis criteria and for whom vasopressors were initiated and lactate values were greater than 2 mmol/L were assigned to the septic shock group. Patients not meeting infection or sepsis criteria were included in the control group.

Patient outcome classifications were assigned by an automated algorithm applied to EHR data. Two physicians on the study team performed a non-blinded chart review of a subset of 100 patients with sepsis (inclusive of septic shock) and 100 patients without sepsis (infection or control). (Liu et al. J Biomed Inform. 2021; 121:103879.) The review with adjudication was conducted to assess the reliability and accuracy of the algorithm with respect to the above definitions. The review resulted in confirmation of reliable classification of sepsis with a positive predictive value (PPV) of 99% and negative predictive value (NPV) of 100%.

Analysis. Continuous variables were expressed as median with interquartile range (IQR) and compared using the Mann-Whitney U test. Categorical variables were expressed as number and percentages and were compared using the χ2 test. Correlation coefficients were calculated using the Spearman rank method. Diagnostic performance was evaluated using binary classification measures. The area under the receiver operator characteristic curve (AUC) was calculated using logistic regression models with sepsis (sepsis without shock or septic shock) and septic shock as separate response variables. Leukocyte parameters were modeled in isolation (single predictor) and in combination (multiple predictors) as continuous variables. Comparisons of the AUC and their CIs were evaluated using the De Long method. (DeLong et al. Biometrics 1988; 44 (3): 837-845.) Sensitivity, specificity, positive and negative predictive value and likelihood ratios were calculated using laboratory cutoffs with definitions for dichotomization as normal or abnormal. Patients with missing lactate or CRP were excluded from respective subgroup analyses. No imputation or interpolation methods were applied to any clinical data used to derive sepsis outcomes. All analysis was performed in Python Version 3.

TABLE 1
Study Patient Characteristics

| | Total | Control | Infection | Sepsis | Septic Shock |
|---|---|---|---|---|---|
| Patients, No. | 7952 | 6597 | 1175 | 137 | 43 |
| Sex Female, No. | 4196 (52.8%) | 3494 (53.0%) | 621 (52.9%) | 54 (39.4%) | 27 (62.8%) |
| (%) | | | | | |
| Age, Median | 50.0 (34.0-63.0) | 49.0 (33.0-62.0) | 53.0 (36.0-65.0) | 62.0 (49.0-69.0) | 66.0 (53.5-73.5) |
| (IQR) [years] | | | | | |
| Race, No. (%) | | | | | |
| Black | 4567 (57.4%) | 3843 (58.3%) | 635 (54.0%) | 64 (46.7%) | 25 (58.1%) |
| White | 2499 (31.4%) | 1999 (30.3%) | 423 (36.0%) | 60 (43.8%) | 17 (39.5%) |
| Other | 886 (11.1%) | 755 (11.4%) | 117 (10.0%) | 13 (9.5%) | 1 (2.3%) |
| Presenting | | | | | |
| Complaints, No. | | | | | |
| (%) | | | | | |
| Abdominal Pain | 802 (10.1%) | 653 (9.9%) | 136 (11.6%) | 10 (7.3%) | 3 (7.0%) |
| Altered Mental | 130 (1.6%) | 100 (1.5%) | 19 (1.6%) | 7 (5.1%) | 4 (9.3%) |
| Status | | | | | |
| Chest Pain | 749 (9.4%) | 698 (10.6%) | 50 (4.3%) | 1 (0.7%) | 0 (0.0%) |
| Dizziness | 166 (2.1%) | 150 (2.3%) | 14 (1.2%) | 0 (0.0%) | 2 (4.7%) |
| Fever | 162 (2.0%) | 89 (1.3%) | 57 (4.9%) | 13 (9.5%) | 3 (7.0%) |
| Generalized | 97 (1.2%) | 72 (1.1%) | 17 (1.4%) | 6 (4.4%) | 2 (4.7%) |
| Weakness | | | | | |
| Hypotension | 46 (0.6%) | 15 (0.2%) | 24 (2.0%) | 4 (2.9%) | 3 (7.0%) |
| Shortness of | 632 (7.9%) | 489 (7.4%) | 118 (10.0%) | 20 (14.6%) | 5 (11.6%) |
| Breath | | | | | |
| Comorbidities, | | | | | |
| No. (%) | | | | | |
| Coronary Artery | 614.0 (7.7%) | 486.0 (7.4%) | 110.0 (9.4%) | 17.0 (12.4%) | 1.0 (2.3%) |
| Disease | | | | | |
| Cancer | 1157.0 (14.5%) | 889.0 (13.5%) | 216.0 (18.4%) | 38.0 (27.7%) | 14.0 (32.6%) |
| Cerebrovascular | 389.0 (4.9%) | 315.0 (4.8%) | 60.0 (5.1%) | 11.0 (8.0%) | 3.0 (7.0%) |
| Disease | | | | | |
| Diabetes | 1057.0 (13.3%) | 819.0 (12.4%) | 200.0 (17.0%) | 31.0 (22.6%) | 7.0 (16.3%) |
| Heart Failure | 475.0 (6.0%) | 368.0 (5.6%) | 82.0 (7.0%) | 18.0 (13.1%) | 7.0 (16.3%) |
| Hypertension | 2030.0 (25.5%) | 1611.0 (24.4%) | 358.0 (30.5%) | 47.0 (34.3%) | 14.0 (32.6%) |
| Immuno- | 850.0 (10.7%) | 614.0 (9.3%) | 189.0 (16.1%) | 38.0 (27.7%) | 9.0 (20.9%) |
| suppression | | | | | |
| Kidney Disease | 831.0 (10.5%) | 593.0 (9.0%) | 204.0 (17.4%) | 26.0 (19.0%) | 8.0 (18.6%) |
| Liver Disease | 897.0 (11.3%) | 719.0 (10.9%) | 154.0 (13.1%) | 18.0 (13.1%) | 6.0 (14.0%) |
| Prior Respiratory | 43.0 (0.5%) | 30.0 (0.5%) | 11.0 (0.9%) | 2.0 (1.5%) | 0.0 (0.0%) |
| Failure | | | | | |
| qSOFA ≥ 2, No. | 94 (1.2%) | 54 (0.8%) | 19 (1.6%) | 11 (8.0%) | 10 (23.3%) |
| % | | | | | |
| Secondary | | | | | |
| Outcomes | | | | | |
| Mortality, No. | 132 (1.7%) | 67 (1.0%) | 37 (3.1%) | 19 (13.9%) | 9 (20.9%) |
| (%) | | | | | |
| Intensive Care, | 239 (3.0%) | 121 (1.8%) | 53 (4.5%) | 36 (26.3%) | 29 (67.4%) |
| No. (%) | | | | | |
| Hospitalization, | 2717 (34.2%) | 1864 (28.3%) | 673 (57.3%) | 137 (100.0%) | 43 (100.0%) |
| No. (%) | | | | | |
| Hospital | 18.3 (8.1-80.3) | 14.6 (7.6-56.8) | 72.9 (13.5-167.5) | 219.4 (149.0-392.3) | 273.6 (156.3-387.9) |
| Duration, Median | | | | | |
| (IQR) [hours] | | | | | |
| Lab Results, | | | | | |
| Median (IQR) | | | | | |
| [Units] | | | | | |
| Monocyte | 18.8 (17.3-21.0) | 18.6 (17.1-20.4) | 20.4 (18.2-23.3) | 23.9 (20.2-26.8) | 24.8 (22.0-28.1) |
| Distribution | | | | | |
| Width [U] | | | | | |
| White Blood | 7.4 (5.6-9.8) | 7.1 (5.6-9.3) | 8.7 (6.2-11.7) | 13.0 (8.9-18.6) | 14.8 (8.7-17.8) |
| Cell, Count [G/L] | | | | | |
| Neutrophil- | 3.0 (1.9-5.4) | 2.8 (1.8-4.7) | 4.4 (2.4-8.5) | 11.2 (6.6-19.6) | 9.6 (5.4-18.8) |
| Lymphocyte Ratio | | | | | |

Abbreviations: qSOFA: quick Sequential Organ Failure Assessment, No: number, IQR: interquartile range, U: Unit

TABLE 2
Diagnostic Performance of Monocyte Distribution Width, White Blood
Cell Count, and Neutrophil-to-Lymphocyte Ratio in isolation and in
combination for Sepsis (with or without shock) and Septic Shock

| | | | | | | | |
|---|---|---|---|---|---|---|---|
| | Definition | | | | | | |
| | for | | | | | | |
| AUC | Positive | Sensitivity % | Specificity % | PPV % | NPV % | | |
| (95% CI) | Test | (95% CI) | (95% CI) | (95% CI) | (95% CI) | LR+ | LR− |

Sepsis (without Shock or Septic Shock)

| | | | | | | | | |
|---|---|---|---|---|---|---|---|---|
| qSOFA | 0.71 | >2 | 52.2 | 88.6 | 9.6 | 98.8 | 4.60 | 0.54 |
| | (0.67-0.75) | | (44.9-59.5) | (87.9-89.4) | (7.8-11.5) | (98.5-99.0) | | |
| MDW | 0.80 | >20 | 77.8 | 66.8 | 5.2 | 99.2 | 2.35 | 0.33 |
| | (0.77-0.84) | | (71.7-83.9) | (65.8-67.9) | (4.3-6.0) | (99.0-99.5) | | |
| WBC | 0.77 | >12 or | 60.6 | 81.3 | 7.0 | 98.9 | 3.25 | 0.48 |
| | (0.73-0.81) | <4 | (53.4-67.7) | (80.5-82.2) | (5.7-8.3) | (98.6-99.1) | | |
| NLR | 0.84 | >10 | 53.3 | 90.8 | 11.9 | 98.8 | 5.81 | 0.51 |
| | (0.81-0.87) | | (46.0-60.6) | (90.2-91.5) | (9.6-14.1) | (98.6-99.1) | | |
| MDW and | 0.83 | both tests | 52.2 | 91.1 | 11.9 | 98.8 | 5.85 | 0.52 |
| WBC | (0.80-0.86) | positive | (44.9-59.5) | (90.4-91.7) | (9.7-14.2) | (98.5-99.1) | | |
| MDW or | | either test | 86.1 | 57.1 | 4.4 | 99.4 | 2.01 | 0.24 |
| WBC | | positive | (81.1-91.2) | (56.0-58.2) | (3.8-5.1) | (99.2-99.7) | | |
| MDW and | 0.85 | both tests | 43.3 | 94.6 | 15.7 | 98.6 | 8.06 | 0.60 |
| NLR | (0.82-0.88) | positive | (36.1-50.6) | (94.1-95.1) | (12.5-18.9) | (98.4-98.9) | | |
| MDW or | | either test | 87.8 | 63.0 | 5.2 | 99.6 | 2.38 | 0.19 |
| NLR | | positive | (83.0-92.6) | (62.0-64.1) | (4.4-6.0) | (99.4-99.7) | | |
| WBC and | 0.85 | both tests | 36.7 | 95.7 | 16.5 | 98.5 | 8.51 | 0.66 |
| NLR | (0.82-0.88) | positive | (29.6-43.7) | (95.2-96.1) | (12.8-20.1) | (98.2-98.8) | | |
| WBC or | | either test | 77.2 | 76.5 | 7.1 | 99.3 | 3.28 | 0.30 |
| NLR | | positive | (71.1-83.3) | (75.5-77.4) | (5.9-8.2) | (99.1-99.5) | | |
| MDW and | 0.86 | all tests | 32.8 | 97.3 | 21.9 | 98.4 | 12.07 | 0.69 |
| WBC and | (0.83-0.89) | positive | (25.9-39.6) | (96.9-97.6) | (16.9-26.8) | (98.1-98.7) | | |
| NLR | | | | | | | | |
| MDW or | | any test | 92.2 | 54.9 | 4.5 | 99.7 | 2.05 | 0.14 |
| WBC or | | positive | (88.3-96.1) | (53.8-56.0) | (3.9-5.2) | (99.5-99.8) | | |
| NLR | | | | | | | | |

Septic Shock

| | | | | | | | | |
|---|---|---|---|---|---|---|---|---|
| qSOFA | 0.82 | >2 | 74.4 | 88.1 | 3.3 | 99.8 | 6.23 | 0.20 |
| | (0.75-0.89) | | (61.4-87.4) | (87.3-88.8) | (2.2-4.4) | (99.7-99.9) | | |
| MDW | 0.85 | >20 | 83.7 | 66.1 | 1.3 | 99.9 | 2.47 | 0.25 |
| | (0.79-0.91) | | (72.7-94.8) | (65.1-67.1) | (0.9-1.8) | (99.8-100.0) | | |
| WBC | 0.79 | >12 or | 65.1 | 80.6 | 1.8 | 99.8 | 3.36 | 0.43 |
| | (0.71-0.87) | <4 | (50.9-79.4) | (79.8-81.5) | (1.1-2.5) | (99.6-99.9) | | |
| NLR | 0.81 | >10 | 46.5 | 90.0 | 2.5 | 99.7 | 4.66 | 0.59 |
| | (0.73-0.88) | | (31.6-61.4) | (89.4-90.7) | (1.4-3.5) | (99.5-99.8) | | |
| MDW or | 0.84 | both tests | 55.8 | 90.3 | 3.0 | 99.7 | 5.78 | 0.49 |
| WBC | (0.77-0.90) | positive | (41.0-70.7) | (89.7-91.0) | (1.8-4.2) | (99.6-99.9) | | |
| MDW and | | either test | 93.0 | 56.4 | 1.1 | 99.9 | 2.13 | 0.12 |
| WBC | | positive | (85.4-100.0) | (55.3-57.5) | (0.8-1.5) | (99.9-100.0) | | |
| MDW or | 0.87 | both tests | 39.5 | 93.9 | 3.4 | 99.7 | 6.53 | 0.64 |
| NLR | (0.82-0.92) | positive | (24.9-54.1) | (93.4-94.5) | (1.8-5.0) | (99.5-99.8) | | |
| MDW and | | either test | 90.7 | 62.2 | 1.3 | 99.9 | 2.40 | 0.15 |
| NLR | | positive | (82.0-99.4) | (61.1-63.3) | (0.9-1.7) | (99.8-100.0) | | |
| WBC or | 0.85 | both tests | 32.6 | 95.1 | 3.5 | 99.6 | 6.65 | 0.71 |
| NLR | (0.78-0.91) | positive | (18.6-46.6) | (94.6-95.6) | (1.7-5.3) | (99.5-99.8) | | |
| WBC and | | either test | 79.1 | 75.6 | 1.7 | 99.8 | 3.24 | 0.28 |
| NLR | | positive | (66.9-91.2) | (74.6-76.5) | (1.2-2.3) | (99.8-99.9) | | |
| MDW or | 0.86 | all tests | 30.2 | 96.8 | 4.8 | 99.6 | 9.30 | 0.72 |
| WBC or | (0.80-0.92) | positive | (16.5-44.0) | (96.4-97.1) | (2.3-7.4) | (99.5-99.7) | | |
| NLR | | | | | | | | |
| MDW and | | any test | 97.7 | 54.1 | 1.1 | 100.0 | 2.13 | 0.04 |
| WBC and | | positive | (93.2-100.0) | (53.0-55.2) | (0.8-1.5) | (99.9-100.0) | | |
| NLR | | | | | | | | |

Abbreviations:
qSOFA: quick Sequential Organ Failure Assessment,
MDW: Monocyte Distribution Width,
WBC: White Blood Count,
NLR: neutrophil-to-lymphocyte ratio,
U: Unit,
AUC: Area Under Curve,
PPV: Positive Predictive Value,
NPP: Negative Predictive Value,
LR+: Positive Likelihood Ratio,
LR−: Negative Likelihood Ratio,
CI: Confidence Interval

### Results

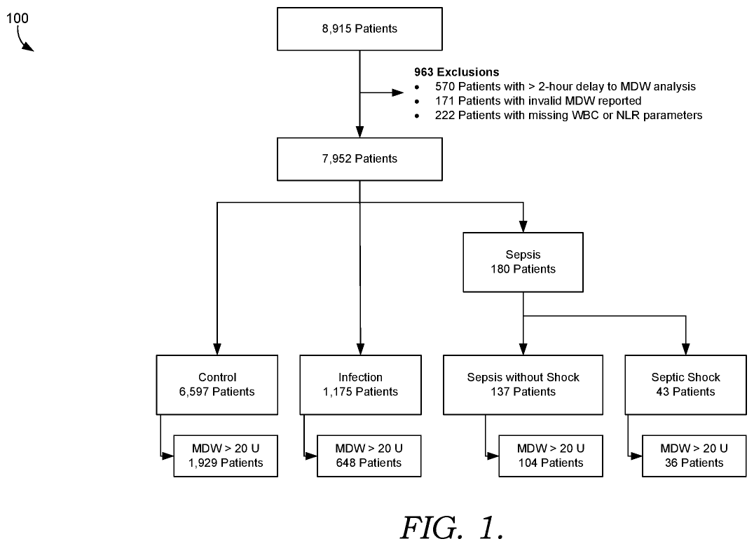

Characteristics of study subjects. A total of 8,915 patients with MDW measured within 6 hours of ED arrival were included in the study. Patients were excluded due to greater than 2-hour delays from blood collection to MDW analysis (570 patients), invalid MDW measurements (171), and missing correlate WBC, neutrophil or lymphocyte counts (222) as seen in chart 100 of FIG. 1. This left a final cohort of 7,952 patients comprised of 6,597 (83.0%) controls, 1,175 (14.8%) patients with infection, 137 (1.7%) meeting criteria for sepsis without shock, and 43 patients (0.5%) meeting criteria for septic shock. Patients meeting infectious disease outcome criteria tended to be older and were more likely to suffer from comorbid conditions such as cancer, heart failure and kidney disease as seen in Table 1. They were also more likely to meet 2 or more qSOFA criteria at presentation, to require hospital or ICU admission and to experience mortality.

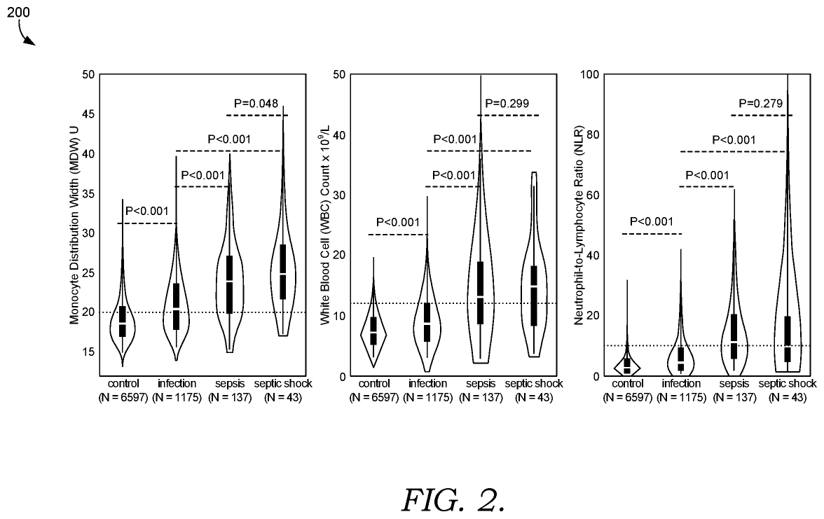

Main results. The distributions of MDW, WBC, and NLR are displayed in FIG. 2. MDW was highest for patients with septic shock (median: 24.8 U, IQR 22.0-28.1) and trended downward for sepsis (23.9 U, IQR 20.2-26.8) and infection (20.4 U, IQR 18.2-23.3) with lowest values observed in control patients (18.6 U, IQR 17.1-20.4) as seen in FIG. 2. WBC and NLR distinguished sepsis from non-sepsis (control and infection) groups but showed less discrimination between sepsis (without shock) and septic shock groups compared to MDW (P=0.048). Further MDW demonstrated low positive correlation with WBC (rho=0.09, 95% CI [0.07-0.11]) and NLR (rho=0.19, 95% CI [0.17-0.22]), while WBC and NLR were moderately correlated (rho=0.52, 95% CI [0.50-0.53]).

The diagnostic performance of qSOFA and leukocyte parameters (MDW, WBC, NLR) for sepsis groups is displayed in Table 2. MDW detected sepsis with an AUC of 0.80 (95% CI 0.77-0.84) and septic shock with an AUC of 0.85 (95% CI 0.79-0.91). In comparison, WBC had an AUC of 0.77 (95% CI 0.73-0.81) and 0.79 (95% CI 0.71-0.87) and NLR had an AUC of 0.84 (95% CI 0.81-0.87) and 0.81 (95% CI 0.73-0.88) for sepsis and septic shock, respectively. Using a cutoff of 20 U or greater for MDW as a test for sepsis shows a sensitivity of 77.8% (95% CI 71.1-83.9) and specificity of 66.8% (95% CI 65.8-67.9%). Overall diagnostic performance (AUC) of MDW and NLR in isolation was superior to qSOFA for sepsis (P<0.05). Combining MDW, WBC, and NLR increased overall diagnostic performance to an AUC of 0.86 (95% CI 0.83-0.89) for sepsis and 0.86 (95% CI 0.80-0.92) for septic shock as seen in Table 2.

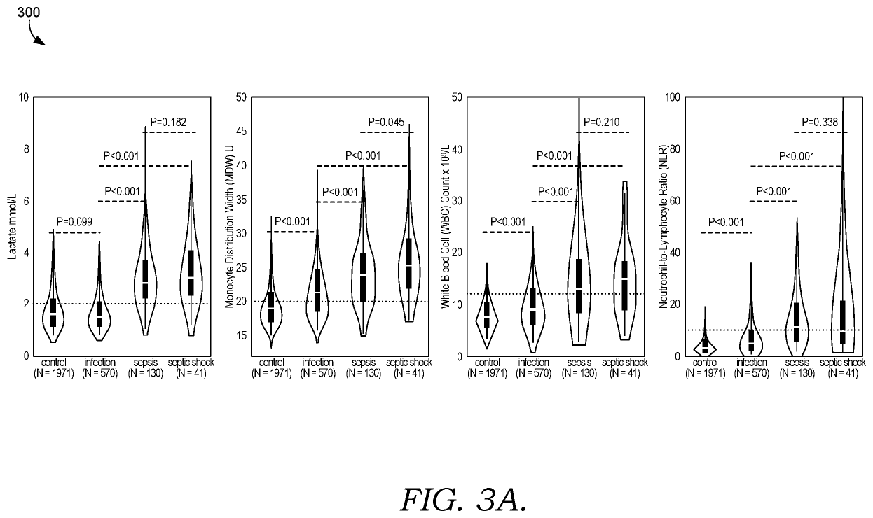

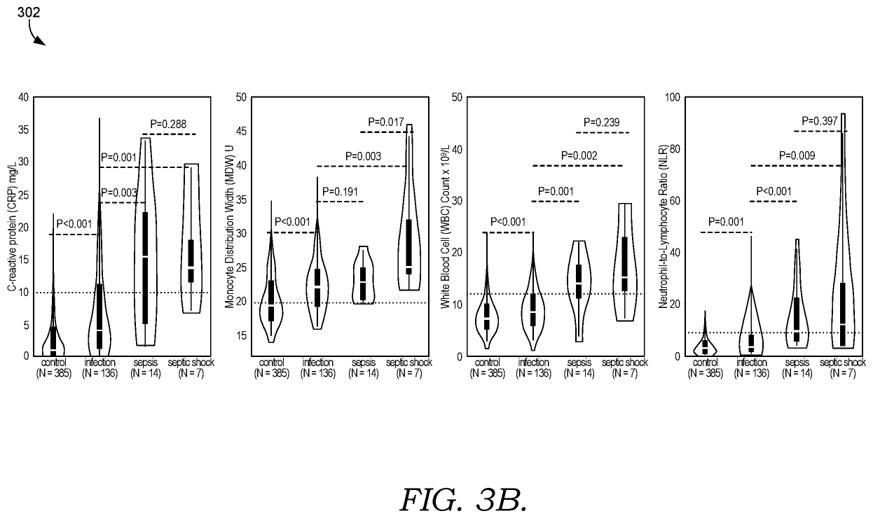

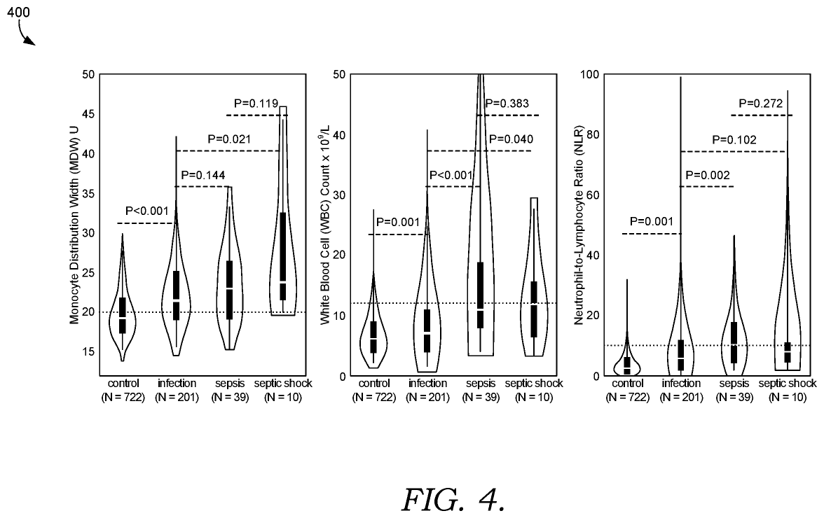

Subgroup Analyses. During routine care in the ED, lactate was measured for a subgroup of 2,712 patients (34.1% of total cohort) and CRP was measured for a subgroup 542 patients (6.8%). FIG. 3A displays the distribution of lactate and FIG. 3B displays the distribution of CRP, in comparison to leukocyte parameters (MDW, WBC, NLR) for these respective subgroups. Lactate demonstrated reliable differences between sepsis and non-sepsis (control and infection) groups with limited differentiation between sepsis (without shock) and septic shock groups as seen in FIG. 3A. CRP in a smaller sample showed similar trends, with no clear differentiation between sepsis (without shock) and septic shock groups (FIG. 3B). MDW maintained its upward trend between control, infection, sepsis, and septic shock in both of these subgroups where additional laboratory work-up was performed. This trend was also maintained for a group of 965 (12.2%) patients meeting criteria for immunosuppression as seen in supplemental FIG. 4.

### DISCUSSION

Despite widespread recognition that early initiation of targeted therapy for sepsis is critical to outcome improvement, rapid identification of patients with the condition remains a major challenge. (Morr et al. BMC Emerg Med. 2017; 17 (1): 11.) Sepsis diagnosis is complicated by vague presentations and a lack of biomarkers or other ancillary tests that reliably rule-in or rule-out the disease. (A1 Jalbout et al. The Journal of Applied Laboratory Medicine. 2019; 3 (4): 724-729; Pierrakos et al. Crit Care. 2020; 24 (1): 287; Boushra et al. J Emerg Med. 2019; 56 (1): 36-45.) Sepsis screening is particularly challenging in the ED, where systemic inflammatory response syndrome (SIRS) and organ failure are often driven by non-infectious pathology and patients with occult infection may present prior to manifesting the tale-tale signs of sepsis (e.g., tachycardia, hypotension, altered mental status) detected by tools like qSOFA that have been applied for screening. (Singer et al. JAMA. 2016; 315 (8): 801-810; Serafim et al. Chest. 2018; 153 (3): 646-655; Anand et al. Chest. 2019; 156 (2): 289-297.)

This Example shows that MDW may have concurrent utility as an ED-based sepsis screen and for severity of illness stratification. In isolation, MDW was the most sensitive marker tested for both sepsis and septic shock, outperforming qSOFA, WBC, and NLR (Table 2). It also demonstrated a NPV of 99.2% for sepsis and 99.9% for septic shock. These results suggest MDW is a strong candidate for broad-based screening where the aim is to identify unsuspected cases of sepsis. In addition, MDW was unique in distinguishing severity of illness (for example, septic shock from sepsis) compared to WBC, NLR, lactate and CRP. For example, NLR which reported the highest discriminatory power for sepsis in this cohort (AUC=0.84) showed limited capability in differentiating sepsis without shock from septic shock; this is consistent with prior findings in higher-risk populations (e.g., ICU). (Farkas, J Thorac Dis. 2020; 12 (Suppl 1): S16-S21.) Thus, complementary clinical attributes of different leukocyte parameters (for example, utility for screening vs risk-stratification) suggests that they are most useful in combination.

When applied together, MDW, WBC, NLR increased the AUC to 0.86 for both sepsis and septic shock (Table 2); a sensitivity of 92.2% for sepsis and 97.7% for septic shock was achieved. These sensitivities translate to negative likelihood ratios of 0.14 and 0.04, respectively. For a patient who presented to the ED with low pretest probability (e.g., 20%), sepsis and septic shock would be effectively ruled out (post-test probabilities 3% and 1%, respectively) by not meeting threshold criteria for any of these three parameters. Further, it is likely that additional precision could be achieved through algorithms that incorporate other clinical data such as patient demographics, medical history, presenting complaint and vital signs; all available prior to CBC results. (Levin et al. Ann Emerg Med. 2018; 71 (5): 565-574.e2.)

The availability of MDW, WBC, and NLR as part of the CBC differential, should not be underestimated. Other markers for sepsis (for example, lactate, CRP and procalcitonin) are commonly employed for sepsis risk-stratification, but none exhibit optimal diagnostic performance when used in isolation and they are not consistently clinically available in all EDs. In contrast, the identification of disease-specific patterns within a routinely used laboratory panel allows for recognition of a clinically time-sensitive disease (sepsis) when it is not suspected, and has potential value in directing interventions to those most at risk of missed or delayed diagnosis and adverse outcome. In this study, MDW was comparable to lactate and CRP in the highly selected group of patients for whom these tests were ordered by treating clinicians (FIG. 3), yet MDW was available for the entire study cohort. WBC, NLR and MDW in isolation exhibited predictive value and unique diagnostic performance characteristics (FIG. 2 and Table 2). However, lack of correlation between MDW with the other CBC parameters indicate that MDW has an important additive role (Crouser et al. Chest. 2017; 152 (3): 518-526) and could help optimize the use of CBC results (Farkas, J Thorac Dis. 2020; 12 (Suppl 1): S16-S21).

This Example describes the largest clinically focused study of MDW to date. The findings support those of several smaller studies that showed MDW in isolation has fair to good accuracy for detection of sepsis in an undifferentiated ED population. (Crouser et al. Crit Care Med. 2019; 47 (8): 1018-1025; Crouser et al. Chest. 2017; 152 (3): 518-526; Crouser et al. J Intensive Care. 2020; 8:33; Agnello et al. Int J Lab Hematol. 2021; 23 (4): 0183; Polilli et al. PLOS ONE. 2020; 15 (1): e0227300; Le et al. Critical Care Medicine. 2020; 48 (1): 12) This study extends those findings by evaluating the performance of MDW alone and in combination with multiple routinely reported components of the CBC to optimize sensitivity and specificity. This Example further describes the performance of MDW relative to both lactate and CRP and to evaluate its performance in a sub-population of patients with immunosuppression. While this sub-analysis was limited by its small sample size, MDW was effective in differentiating patients with infection and sepsis from those without infection, and similar trends in MDW signal were seen in this sample as in the larger population (FIG. 4). These data strengthen the evidence supporting use of MDW for broad-based sepsis screening and argue for its incorporation into comprehensive algorithms for sepsis diagnosis and illness severity estimation. (Le et al. Critical Care Medicine. 2020; 48 (1): 12.)

The complete disclosure of all patents, patent applications, and publications, and electronically available material cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of screening for sepsis or septic shock in a patient, the method comprising:
calculating neutrophil-to-lymphocyte ratio (NLR) for the blood sample from the patient;
characterizing white blood cell count (WBC) for a blood sample;
calculating a monocyte cell population parameter for the blood sample;
comparing NLR to a first predetermined threshold value, WBC to a predetermined threshold range, and the monocyte cell population parameter to a second predetermined threshold value; and
making a sepsis or septic shock determination, wherein making the sepsis or septic shock determination comprises making one or more determinations selected from:
determining whether the patient has an increased risk of sepsis or septic shock; and
determining whether the patient is not at risk of sepsis or septic shock.

2. The method of claim 1, wherein making the sepsis or septic shock determination comprises determining whether the patient has an increased risk of sepsis or septic shock, and wherein determining whether the patient has an increased risk of sepsis or septic shock comprises determining
NLR is greater than the first predetermined threshold value,
WBC is outside of the predetermined threshold range, or
the monocyte cell population parameter is greater than the second predetermined threshold value,
or a combination thereof.

3-5. (canceled)

6. The method of claim 1, wherein the monocyte cell population parameter comprises monocyte distribution width (MDW), wherein the second predetermined threshold value is 20, and wherein determining whether the patient has an increased risk of sepsis or septic shock comprises determining MDW is greater than 20.

7. The method of claim 1 wherein the threshold range is 4×10⁹ cells/L to 12×10⁹ cells/L, and wherein determining whether the patient has an increased risk of sepsis or septic shock comprises determining WBC is less than 4×10⁹ cells/L or greater than 12×10⁹ cells/L.

8. The method of claim 1, wherein the first predetermined threshold value is 10, wherein determining whether the patient has an increased risk of sepsis or septic shock comprises determining NLR is greater than 10.

9. The method of claim 1, wherein the patient presents with a non-specific complaint.

10. The method of claim 1, wherein the patient presents with a symptom of a systemic inflammatory condition.

11. The method of claim 1, wherein the patient presents to an emergency department.

12. (canceled)

13. The method of claim 1, wherein the method further comprises administering a treatment for sepsis wherein administering the treatment for sepsis comprises administering one or more of an antibiotic preparation, an intravenous fluid, a vasopressor, a corticosteroid, insulin, an analgesic, a sedative, or an immune enhancing therapy.

14. (canceled)

15. The method of claim 13, where the method further comprises determining the success of the treatment for sepsis.

16. The method of claim 1, wherein the method further comprises
discretizing one or more of NLR, WBC, and the monocyte cell population parameter to provide a discretized value for one or more of NLR, WBC, and the monocyte cell population parameter,
analyzing the discretized value for one or more of NLR, WBC, and the monocyte cell population with a set of decision rules,
defining weights for a points system based on an odds ratio, and
producing index points for one or more of NLR, WBC, and the monocyte cell population parameter that may be summed to provide an index score.

17-18. (canceled)

19. The method of claim 1, wherein making the sepsis or septic shock determining comprises determining whether the patient is not at risk of sepsis or septic shock, and wherein determining whether the patient is not at risk of sepsis or septic shock comprises determining
NLR is less than a first predetermined threshold value; and
WBC is inside of a predetermined threshold range, or the monocyte cell population parameter is less than the second predetermined threshold value, or both.

20-29. (canceled)

30. A method comprising an automated method for screening for sepsis or septic shock in a patient, the method comprising:
calculating neutrophil-to-lymphocyte ratio (NLR) for a blood sample from the patient;
characterizing white blood cell count (WBC) for the blood sample;
calculating a monocyte cell population parameter for the blood sample; and
determining whether the patient has an increased risk of sepsis or septic shock using a data processing module, wherein the data processing module comprises a processor and a tangible non-transitory computer readable medium, and the computer readable medium is programmed with a computer application that, when executed by the processor, causes the processor to compare NLR to a first predetermined threshold value to provide a first comparison, to compare the monocyte cell population parameter to a second predetermined threshold value to provide a second comparison, to compare WBC to a third predetermined threshold value and a fourth predetermined threshold value to provide a third comparison, and to determine the risk of sepsis or septic shock based on the first comparison, the second comparison, and the third comparison.

31. The method of claim 30, wherein determining the risk of sepsis or septic shock indicates suspicion of sepsis or septic shock if
the first comparison is higher than the first predetermined threshold value,
the second comparison is higher than the second predetermined threshold value, and
the third comparison is either lower than the third predetermined threshold value or higher than the fourth predetermined threshold value.

32. The method of claim 30, wherein the first predetermined threshold value is 10.

33. (canceled)

34. The method of claim 30, wherein the monocyte cell population parameter comprises monocyte distribution width (MDW), and wherein the second predetermined threshold value is 20.

35. The method of claim 30, wherein the third predetermined threshold value is 12×10⁹ cells/L and the fourth predetermined threshold value is 4×10⁹ cells/L.

36. The method of claim 30, further comprising altering a test reporting process based on the evaluation of the sepsis status.

37. The method of claim 30, further comprising treating the individual from whom the blood sample was obtained if the sepsis status indicates sepsis, wherein treating the individual from whom the blood sample was obtain comprises administering one or more of an antibiotic preparation, an intravenous fluid, a vasopressor, a corticosteroid, insulin, an analgesic, a sedative, or an immune enhancing therapy.

38. The method of claim 34, further comprising:
delivering a hydrodynamically focused stream of the blood sample toward a cell interrogation zone of an optical element; and
measuring, with an electrode assembly, current (DC) impedance of cells of the blood sample passing individually through the cell interrogation zone; and
wherein the module determines MDW based on the DC impedance measurement of cells of the blood sample.
